(19) United States
(12) Patent Application Publication
(10) Pub. No.: US 2020/0048237 A1
(43) Pub. Date: Feb. 13, 2020

(54) A Free Base Oxazine Derivative in Crystalline Form

(71) Applicant: NOVARTIS AG, Basel (CH)

(72) Inventor: Rita Ramos, Basel (CH)

(21) Appl. No.: 16/478,736

(22) PCT Filed: Jan. 18, 2018
(86) PCT No.: PCT/IB2018/050314

§ 371 Date: Jul. 17, 2019

(30) Foreign Application Priority Data
Jan. 20, 2017 (EP) 17152481.2

(51) Int. Cl. C07D 413/14; A61P 25/28

(52) U.S. Cl. CPC C07D 413/14; A61P 25/28

(57) ABSTRACT

The invention relates to a solid form, namely crystalline Form A, of Compound 1, (1) and discloses the process for making said solid form of Compound 1. Also disclosed are further solid forms of Compound 1, including its hydrate and amorphous form. The present invention further relates to a pharmaceutical composition comprising crystalline Form A of Compound 1, and methods of using said form and pharmaceutical composition in the treatment or prevention of Alzheimer's disease or cerebral amyloid angiopathy.

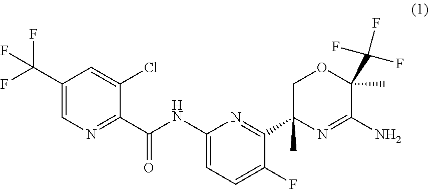 [SMILES: C[C@@]1(c2nc(NC(=O)c3ncc(C(F)(F)F)cc3Cl)ccc2F)CO[C@@](C)(C(F)(F)F)C(N)=N1]

### FIELD OF THE INVENTION

The present invention relates to a specific solid form of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, namely Form A. The present invention further discloses the process for preparing said solid form, pharmaceutical compositions comprising said solid form, and methods of using said solid form and pharmaceutical compositions in the treatment or prevention of Alzheimer's disease or cerebral amyloid angiopathy.

### BACKGROUND

Alzheimer's disease (AD) is one of the most prevalent neurological disorders worldwide and the most common and debilitating age-related condition, causing progressive amnesia, dementia, and ultimately global cognitive failure and death. Currently, the only pharmacological therapies available are symptomatic drugs such as cholinesterase inhibitors or other drugs used to control the secondary behavioural symptoms of AD. Investigational treatments targeting the AD pathogenic cascade include those intended to interfere with the production, accumulation, or toxic sequelae of amyloid-β (Aβ) species (Kramp V P, Herrling P, 2011). Strategies that target decreasing Aβ by: (1) enhancing the amyloid clearance with an active or passive immunotherapy against Aβ; (2) decreasing production through inhibition of Beta-site-APP cleaving enzyme-1 (BACE-1, an enzyme involved in the processing of the amyloid precursor protein [APP]), are of potential therapeutic value. No effective disease-modifying treatment of AD has yet been described in the literature.

Cerebral amyloid angiopathy (CAA) is a common age related cerebral small vessel disease, characterised by progressive deposition of amyloid-β (Aβ), in particular Aβ40, in the wall of small to medium sized arteries, arterioles and capillaries of the cerebral cortex and overlying leptomeninges (Charidimou A et al., 2011). CAA often coexists with Alzheimer's disease (AD). Mild forms of CAA often appear asymptomatic; however, CAA may also lead to severe vascular pathologies and is a risk factor for cerebral hemorrhages ranging from silent microbleeds to spontaneous intracerebral haemorrhage, a devastating form of stroke.

APOE4 is a strong genetic risk factor for both AD and CAA (Shinohara M et al., 2016). Human ApoE is located on chromosome 19 (gene APOE, Uniprot P02649). Three major isoforms (apoE2, -3 and -4) are known in humans. ApoE4 (with Arg at positions 112 and 158) has an allele frequency of 5-35% in humans (Verghese P B et al., 2011) and ApoE4 homozygotes are estimated to represent about 2 to 3% of the general population (Quintino-Santos S R et al., 2012).

Strategies that target decreasing Aβ by: (1) enhancing the amyloid clearance with an active or passive immunotherapy against Aβ; (2) decreasing production through inhibition of Beta-site-APP cleaving enzyme-1 (BACE-1, an enzyme involved in the processing of the amyloid precursor protein [APP]), are of potential therapeutic value in the treatment or prevention of AD and CAA, particularly in patients carrying one or two copies of the ApoE4 allele.

N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, hereafter referred to as Compound 1,

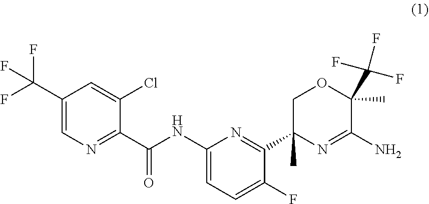 [SMILES: C[C@@]1(c2nc(NC(=O)c3ncc(C(F)(F)F)cc3Cl)ccc2F)CO[C@@](C)(C(F)(F)F)C(N)=N1]

is an orally active BACE inhibitor, previously described in WO 2012/095469 A1. However, after a specific compound is identified as a promising candidate for use in a pharmaceutical composition, it is still necessary to fine-tune its properties with respect to a number of critical parameters, such as stability in solid state and/or liquid formulations, hygroscopicity, crystallinity, toxicological considerations, melting point, or solubility in water and aqueous media.

There is thus a need for solid forms of Compound 1, for use in drug substance and drug product development. It has been found that solid forms of Compound 1 can be prepared as one or more polymorph forms, including a hydrate form. These polymorph forms exhibit physical properties that may be exploited in order to further improve pharmacological properties, and that may be utilized in drug substance and drug product development.

### SUMMARY OF THE INVENTION

In one aspect, provided herein is a crystalline Form A of the Compound of Formula 1.

In another aspect, provided herein is a pharmaceutical composition comprising the crystalline Form A of the Compound of Formula 1 and at least one pharmaceutically acceptable carrier or diluent.

In another aspect, provided herein is the crystalline Form A of the Compound of Formula 1 for use as a medicament.

In a further aspect, provided herein is the crystalline Form A of the Compound of Formula 1 for use in the treatment or prevention of Alzheimer's disease or cerebral amyloid angiopathy.

### BRIEF DESCRIPTION OF THE DRAWINGS

FIG. 1 shows the X-ray powder diffraction pattern for unmicronized crystalline Form A of the Compound of Formula 1 when measured using CuK_{α} radiation.

FIG. 2 shows the DSC thermogram for unmicronized crystalline Form A.

FIG. 3 shows the X-ray powder diffraction pattern for micronized crystalline Form A of the Compound of Formula 1 when measured using CuK_{α} radiation.

FIG. 4 shows the X-ray powder diffraction pattern for crystalline Form B of the Compound of Formula 1 when measured using CuK_{α} radiation.

FIG. 5 shows the DSC thermogram for crystalline Form B.

FIG. 6 shows the X-ray powder diffraction pattern for hydrate Form H_{A} of the Compound of Formula 1 when measured using CuK_{α} radiation.

FIG. 7 shows the DSC thermogram for hydrate Form H_{A} of the Compound of Formula 1.

FIG. 8 shows the X-ray powder diffraction pattern for amorphous form of the Compound of Formula 1 when measured using CuK_{α} radiation.

FIG. 9 shows the mDSC thermogram for amorphous form of the Compound of Formula 1.

FIG. 10 shows the design of a two part, open-label, two-period, fixed-sequence study in healthy subjects to evaluate the PK of Compound 1 when given alone and in combination with the strong CYP3A4 inhibitor itraconazole or the strong CYP3A4 inducer rifampicin.

### DETAILED DESCRIPTION OF THE INVENTION

### Polymorph Forms and Properties

The present invention provides a polymorphic form of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, which is Form A. N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, also referred to as the “Compound of Formula 1” or “Compound 1”, was originally described in WO 2012/095469 A1, Example 34. WO 2012/095469 A1 is incorporated herewith by reference in its entirety, in particular the disclosure related to the synthesis of Example 34.

As described herein, the free base of Compound 1 can be a crystalline form that exists as one or more polymorph forms, including hydrate forms. These polymorph forms (alternatively known in the art as polymorphic forms or crystal forms) differ with respect to their X-ray powder diffraction patterns, spectroscopic, physicochemical and pharmacokinetic properties, as well as their thermodynamic stability.

It is desirable to have access to different polymorphic forms of Compound 1 for several reasons. Distinct polymorph forms may exhibit different physical properties such as melting point, hygroscopicity, solubility, flow properties or thermodynamic stability, and therefore, distinct polymorph forms allow the choice of the most suitable form for a given use or aspect, for example, in distinct administration forms such as capsules, or in the manufacture of a drug form having optimum pharmacokinetic properties.

It has now been surprisingly found that under certain conditions new solid forms of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide can be provided which are described hereinafter as Form A, Form B, hydrate Form H_{A}, and amorphous form, and which have advantageous utilities and properties. In particular, Form A of the Compound of Formula 1 shows excellent stability properties when subject to stress conditions. A particular polymorph form of Compound 1, namely Form A, is more stable than all other solid forms of Compound 1 disclosed herein. This high degree of stability of Form A provides advantageous properties and benefits in terms of its suitability for use in a pharmaceutical composition, for example, in terms of its shelf-life and ease of manufacture.

Reduction of particle size through milling or micronization increases specific surface area, leading to enhanced dissolution and improved homogeneity of the bulk material. Thus, also provided herein is micronized crystalline Form A of the Compound of Formula 1.

The invention provides the crystalline Form A of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide (Compound 1) in free form. The term “free form” refers to the compound per se without salt formation.

Also disclosed herein are Form B, hydrate Form H_{A} and amorphous form in free form.

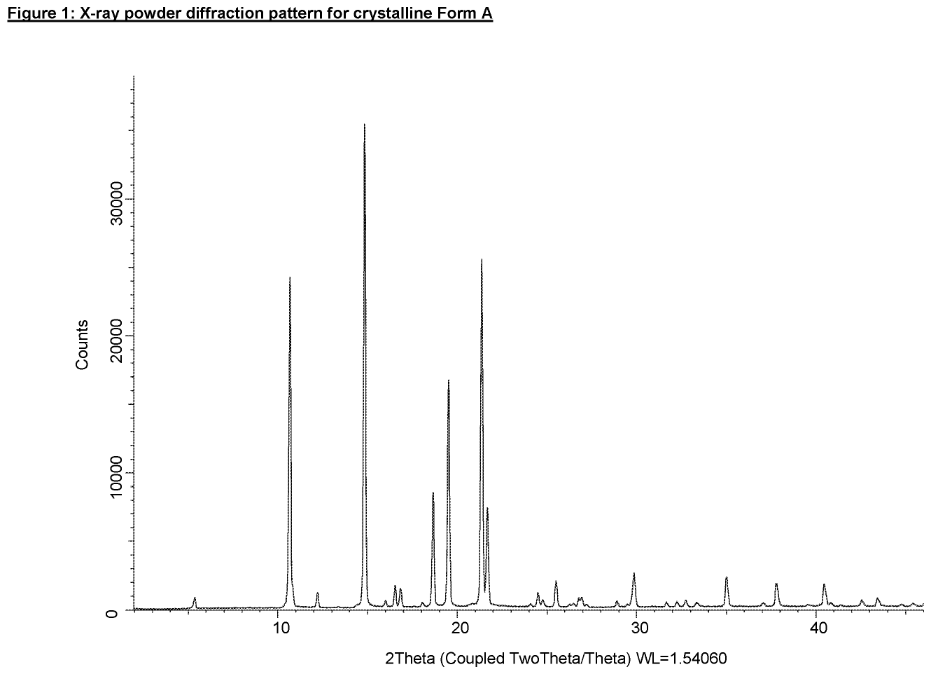

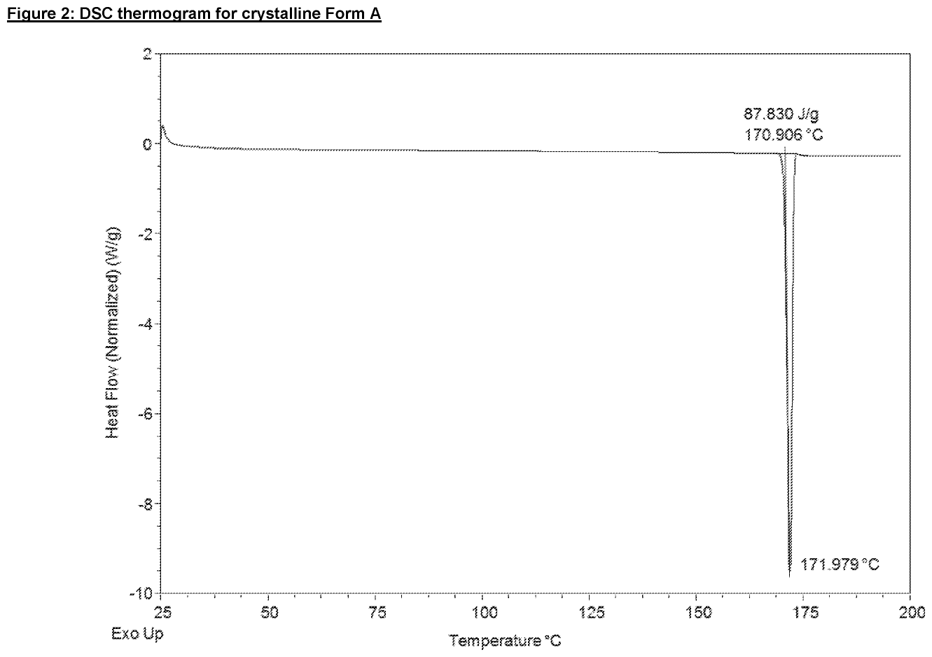

In one embodiment, the Compound of Formula 1 is crystalline Form A. Crystalline Form A can be defined by reference to one or more characteristic signals that result from analytical measurements including, but not limited to: X-ray powder diffraction pattern of FIG. 1, the differential scanning calorimetry (DSC) thermogram of FIG. 2. Crystalline Form A (also referred to herein as polymorph Form A) can also be defined by reference to one or more of the following characteristic signals:

In one embodiment, the crystalline Form A has an X-ray powder diffraction pattern with at least one, two or three peaks having angle of refraction 2 theta (θ) values selected from 14.8, 18.7 and 19.5° when measured using CuKα radiation, wherein said values are plus or minus 0.2° 2θ.

In one embodiment, the crystalline Form A has an X-ray powder diffraction pattern with at least one, two or three peaks having angle of refraction 2 theta (θ) values selected from 10.7, 14.8 and 19.5° when measured using CuKα radiation, wherein said values are plus or minus 0.2° 2θ.

In one embodiment, the crystalline Form A has an X-ray powder diffraction pattern with at least one, two or three peaks having angle of refraction 2 theta (θ) values selected from 10.7, 14.8, 18.7, 19.5 and 21.4° when measured using CuKα radiation, wherein said values are plus or minus 0.2° 2θ.

In one embodiment, the crystalline Form A has an X-ray powder diffraction pattern with at least one, two, three, four or five peaks having angle of refraction 2 theta (θ) values selected from 10.7, 14.8, 18.7, 19.5, 21.4, 21.7, 25.5, 29.9, 35.0, 37.8° when measured using CuKα radiation, wherein said values are plus or minus 0.2° 2θ.

In one embodiment, crystalline Form A of the Compound of Formula 1 exhibits an X-ray powder diffraction pattern substantially the same as the X-ray powder diffraction pattern shown in FIG. 1 when measured using CuKα radiation.

In a further embodiment, crystalline Form A of the Compound of Formula 1 exhibits a differential scanning calorimetry (DSC) thermogram substantially the same as that shown in shown in FIG. 2.

In a further embodiment, crystalline Form A of the Compound of Formula 1 exhibits a differential scanning calorimetry (DSC) thermogram with an onset of melting of about 171° C.

In one embodiment of the invention, there is provided crystalline Form A of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide in substantially pure form.

As used herein, “substantially pure,” when used in reference to crystalline, including hydrate of the crystalline forms and amorphous form of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, means having a purity greater than 90 weight %, including greater than 90, 91, 92, 93, 94, 95, 96, 97, 98, and 99 weight %, and also including equal to about 100 weight % of N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide, based on the weight of the compound.

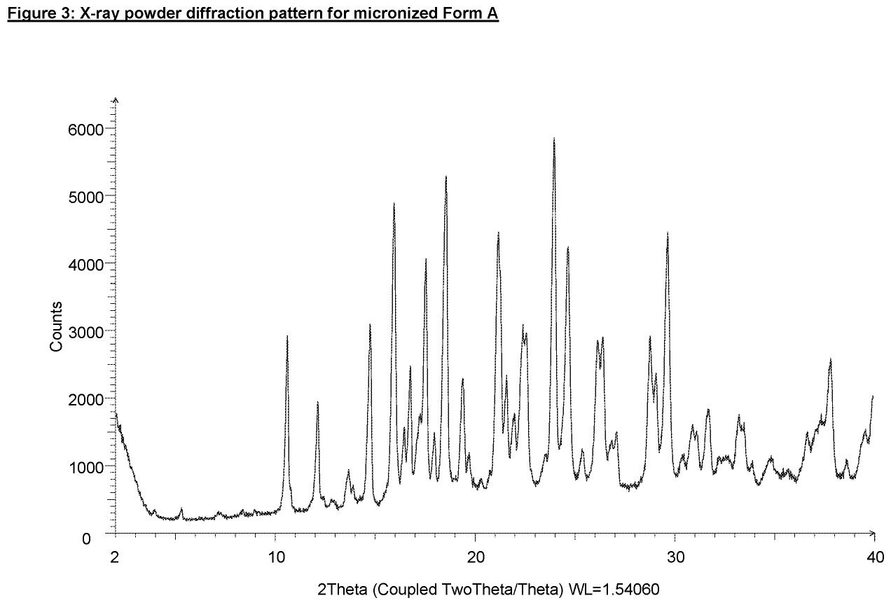

In another embodiment, the Compound of Formula 1 is micronized crystalline Form A. Micronized crystalline Form A can be defined by reference to one or more characteristic signals that result from analytical measurements including, but not limited to: X-ray powder diffraction pattern of FIG. 3. Micronized crystalline Form A can also be defined by reference to one or more of the following characteristic signals: In one embodiment, the micronized crystalline Form A has an X-ray powder diffraction pattern with at least one, two or three peaks having angle of refraction 2 theta (θ) values selected from 12.1, 19.4, 24.0° when measured using CuKα radiation, wherein said values are plus or minus 0.2° 2θ.

In one embodiment, the micronized crystalline Form A has an X-ray powder diffraction pattern with at least one, two or three peaks having angle of refraction 2 theta (θ) values selected from 12.1, 15.9, 18.5, 19.4, 24.0° when measured using CuKα radiation, wherein said values are plus or minus 0.2° 2θ.

In one embodiment, the micronized crystalline Form A has an X-ray powder diffraction pattern with at least one, two, three, four or five peaks having angle of refraction 2 theta (θ) values selected from 10.6, 12.1, 14.7, 15.9, 18.5, 19.4, 21.2, 24.0, 24.7, 29.7° when measured using CuKα radiation, wherein said values are plus or minus 0.2° 2θ.

In one embodiment, micronized crystalline Form A of the Compound of Formula 1 exhibits an X-ray powder diffraction pattern substantially the same as the X-ray powder diffraction pattern shown in FIG. 3 when measured using CuKα radiation.

The term “substantially the same” with reference to X-ray diffraction peak positions means that typical peak position and intensity variability are taken into account. For example, one skilled in the art will appreciate that the peak positions (2θ) will show some inter-apparatus variability, typically as much as 0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. An expression referring to a crystalline Form A having “an X-ray powder diffraction pattern substantially the same as the X-ray powder diffraction pattern shown in Figure X” may be interchanged with an expression referring to a crystalline Form A having “an X-ray powder diffraction pattern characterised by the representative X-ray powder diffraction pattern shown in Figure X”.

One of ordinary skill in the art will also appreciate that an X-ray diffraction pattern may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, and such degree of measurement error should be taken into account as pertaining to the aforementioned diffraction angles. Consequently, it is to be understood that the crystal form of the instant invention is not limited to the crystal form that provides an X-ray diffraction pattern completely identical to the X-ray diffraction pattern depicted in the accompanying FIG. 1 disclosed herein. Any crystal forms that provide X-ray diffraction patterns substantially identical to that disclosed in the accompanying FIG. 1 fall within the scope of the present invention. The ability to ascertain substantial identities of X-ray diffraction patterns is within the purview of one of ordinary skill in the art.

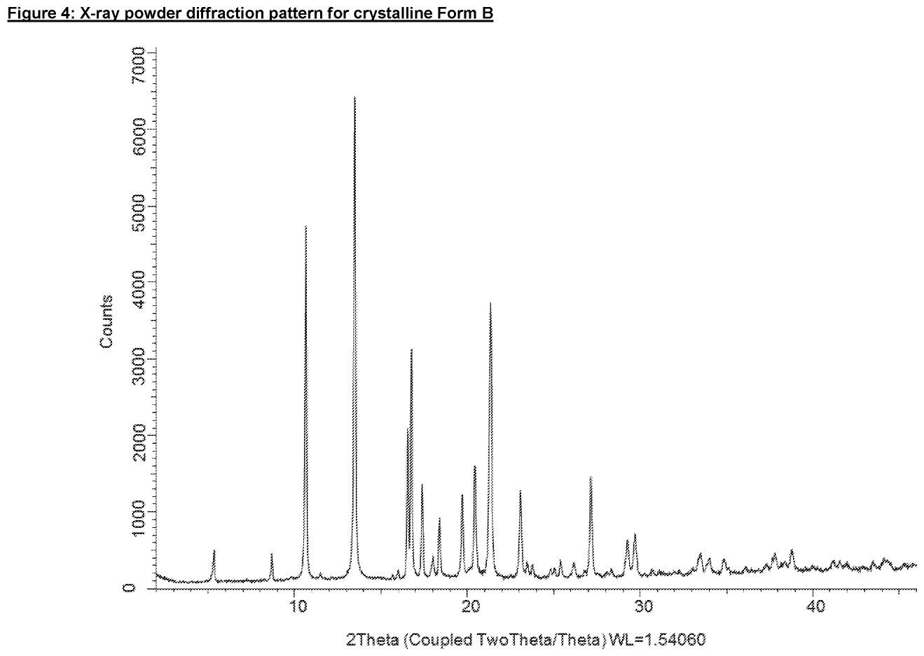

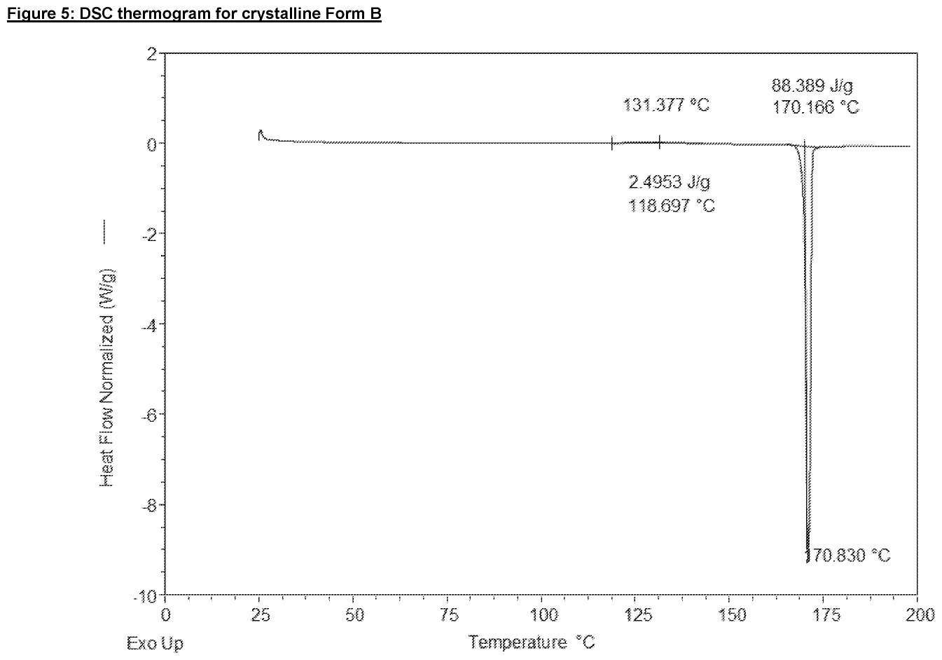

Crystalline Form B can be defined by reference to one or more characteristic signals that result from analytical measurements including, but not limited to: X-ray powder diffraction pattern of FIG. 4, the differential scanning calorimetry (DSC) thermogram of FIG. 5. Crystalline Form B (also referred to herein as polymorph Form B) can also be defined by reference to one or more of the following characteristic signals: The crystalline Form B has an X-ray powder diffraction pattern with at least one, two or three peaks having angle of refraction 2 theta (θ) values selected from 13.5, 20.5, 23.1° when measured using CuKα radiation, wherein said values are plus or minus 0.2° 2θ.

The crystalline Form B has an X-ray powder diffraction pattern with at least one, two or three peaks having angle of refraction 2 theta (θ) values selected from 10.7, 13.5, 16.6, 20.5, 23.1° when measured using CuKα radiation, wherein said values are plus or minus 0.2° 2θ.

The crystalline Form B has an X-ray powder diffraction pattern with at least one, two, three, four or five peaks having angle of refraction 2 theta (θ) values selected from 10.7, 13.5, 16.6, 16.8, 17.4, 19.7, 20.5, 21.3, 23.1, 27.2° when measured using CuKα radiation, wherein said values are plus or minus 0.2° 2θ.

The crystalline Form B of the Compound of Formula 1 exhibits an X-ray powder diffraction pattern substantially the same as the X-ray powder diffraction pattern shown in FIG. 4 when measured using CuKα radiation.

The crystalline Form B of the Compound of Formula 1 exhibits a differential scanning calorimetry (DSC) thermogram substantially the same as that shown in shown in FIG. 5.

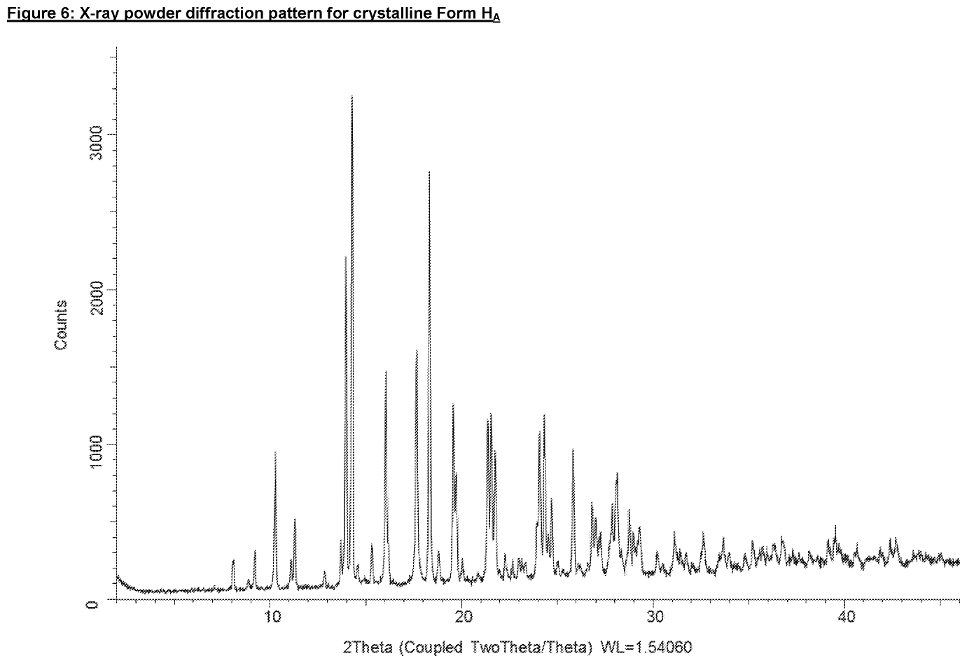

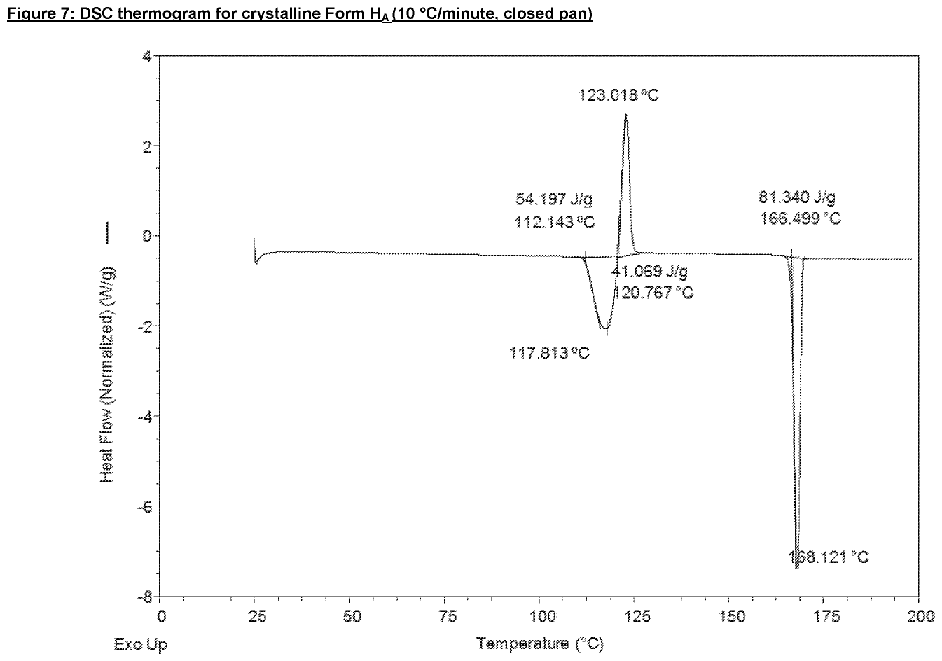

Crystalline hydrate form H_{A} can be defined by reference to one or more characteristic signals that result from analytical measurements including, but not limited to: X-ray powder diffraction pattern of FIG. 6, the differential scanning calorimetry (DSC) thermogram of FIG. 7. Crystalline hydrate form H_{A} (also referred to herein as hydrate form H_{A}) can also be defined by reference to one or more of the following characteristic signals: The hydrate form H_{A} has an X-ray powder diffraction pattern with at least one, two or three peaks having angle of refraction 2 theta (θ) values selected from 14.0, 14.3, 18.3° when measured using CuKα radiation, wherein said values are plus or minus 0.2° 2θ.

The hydrate form H_{A} has an X-ray powder diffraction pattern with at least one, two or three peaks having angle of refraction 2 theta (θ) values selected from 14.0, 14.3, 16.1, 17.7, 18.3° when measured using CuKα radiation, wherein said values are plus or minus 0.2° 2θ.

The hydrate form H_{A} has an X-ray powder diffraction pattern with at least one, two, three, four or five peaks having angle of refraction 2 theta (θ) values selected from 14.0, 14.3, 16.1, 17.7, 18.3, 19.6, 21.4, 21.6, 24.1, 25.8° when measured using CuKα radiation, wherein said values are plus or minus 0.2° 2θ.

The hydrate form H_{A} of the Compound of Formula 1 exhibits an X-ray powder diffraction pattern substantially the same as the X-ray powder diffraction pattern shown in FIG. 6 when measured using CuKα radiation.

The hydrate form H_{A} of the Compound of Formula 1 exhibits a differential scanning calorimetry (DSC) thermogram substantially the same as that shown in shown in FIG. 7.

As used herein, the term “hydrate” refers to a molecular complex of Compound 1 with one or more water molecules. This also encompasses the hemihydrate form, which is defined as a hydrate in which the molecular ratio of water molecule(s) to anhydrous compound is 1:2. For example, the hydrate Form H_{A} is a hemihydrate.

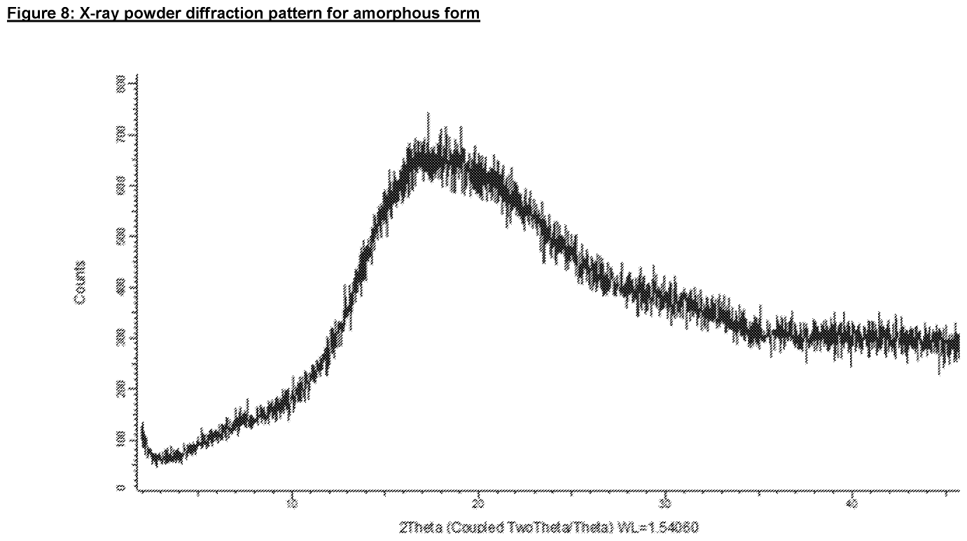

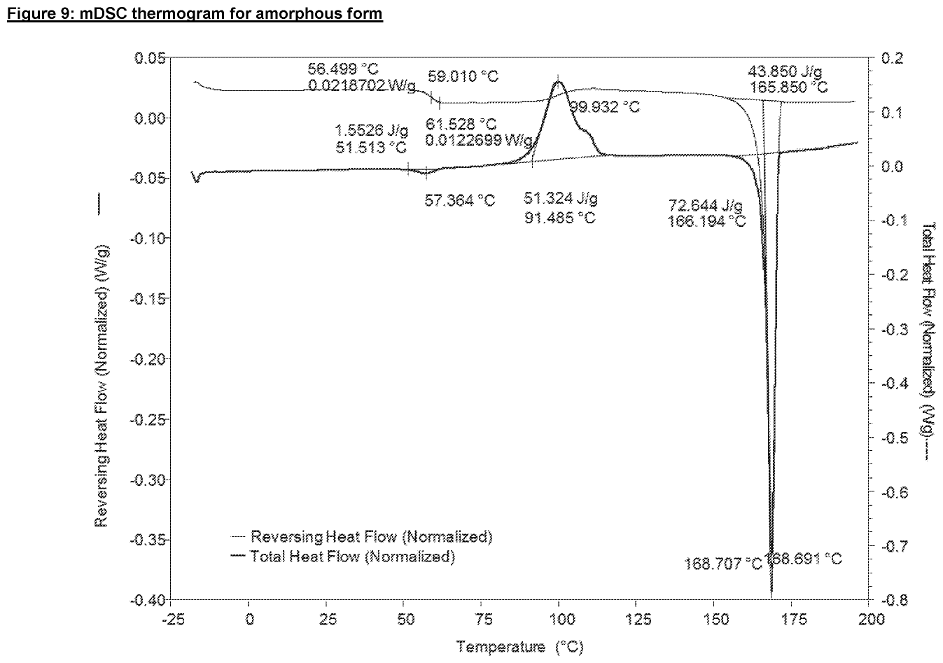

The amorphous form can be defined by analytical measurements including, but not limited to: reference to an X-ray powder diffraction pattern substantially the same as the X-ray powder diffraction pattern shown in FIG. 8 and the modulated differential scanning calorimetry (mDSC) thermogram of FIG. 9.

Seed crystals may be added to any crystallization mixture to promote crystallization. Seeding may be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369-377. In general, seeds of small size are needed to control effectively the growth of crystals in the batch. Seed of small size may be generated by sieving, milling, or micronizing of large crystals, or by micro-crystallization of solutions. Care should be taken that milling or micronizing of crystals does not result in any change in crystallinity form the desired crystal form (i.e., change to amorphous or to another polymorph).

### Method of Treatment

The present invention also provides a method for the treatment or prevention of diseases, conditions and/or disorders modulated by BACE inhibition, for example such as indicated herein, in a subject in need of such treatment or prevention, which method comprises administering to said subject an effective amount of a Compound of Formula 1, especially polymorph Form A.

In one embodiment of the method, the BACE inhibition is inhibition of BACE-1.

In another embodiment of the method, the disease or disorder is Alzheimer's disease or cerebral amyloid angiopathy.

In one embodiment, the present invention provides the use of crystalline Form A of the Compound of Formula 1 for the manufacture of a medicament for the treatment or prevention of Alzheimer's disease or cerebral amyloid angiopathy.

In another aspect, provided herein is crystalline Form A of the Compound of Formula 1 for use as a medicament.

In a further aspect, provided herein is crystalline Form A of the Compound of Formula 1 for use in the treatment or prevention of Alzheimer's disease or cerebral amyloid angiopathy.

### Pharmaceutical Compositions

The Compound of Formula 1, especially polymorph Form A is suitable as an active agent in pharmaceutical compositions that are efficacious particularly for the treatment or prevention of diseases, conditions and/or disorders modulated by BACE inhibition, for example, Alzheimer's disease or cerebral amyloid angiopathy. The pharmaceutical composition in various embodiments has a pharmaceutically effective amount of the crystalline Compound of Formula 1, especially the polymorph Form A, along with one or more pharmaceutically acceptable carriers.

As used herein, a “pharmaceutical composition” comprises Form A and at least one pharmaceutically acceptable carrier, in a unit dose solid form suitable for oral administration (typically a capsule, more particularly a hard gelatin capsule). A list of pharmaceutically acceptable carriers can be found in Remington's Pharmaceutical Sciences. Examples of suitable formulations for Form A are provided in Examples 6 and 7.

Thus, in one aspect, provided herein is a pharmaceutical composition comprising polymorph Form A of the Compound of Formula 1. In one embodiment, the pharmaceutical composition comprises the polymorph Form A of the Compound of Formula 1 and at least one pharmaceutically acceptable carrier.

### Definitions

As used herein, the terms “Compound 1”, “Cmpd 1”, “Compound of Formula 1” refer to N-(6-((3R,6R)-5-amino-3,6-dimethyl-6-(trifluoromethyl)-3,6-dihydro-2H-1,4-oxazin-3-yl)-5-fluoropyridin-2-yl)-3-chloro-5-(trifluoromethyl)picolinamide and having the following structural formula:

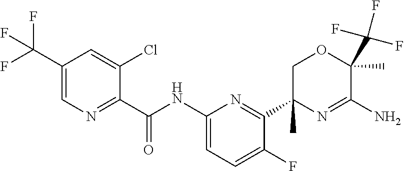 [SMILES: C[C@@]1(c2nc(NC(=O)c3ncc(C(F)(F)F)cc3Cl)ccc2F)CO[C@@](C)(C(F)(F)F)C(N)=N1]

In Example 1, using an alternative chemical naming format, “Compound 1” is also referred to as 3-chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide.

As used herein, “crystalline Form A”, “polymorph Form A” and “Form A” are used interchangeably and have no difference in meaning.

As used herein, the term “pharmaceutically acceptable carrier” includes any and all solvents, dispersion media, coatings, surfactants, antioxidants, preservatives (for example, antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term “Alzheimer's disease” or “AD” encompasses both preclinical and clinical Alzheimer's disease unless the context makes clear that either only preclinical Alzheimer's disease or only clinical Alzheimer's disease is intended.

As used herein, the term “treatment of Alzheimer's disease” refers to the administration of the Compound of Formula 1, especially polymorph Form A, to a patient in order to ameliorate at least one of the symptoms of Alzheimer's disease.

As used herein, the term “prevention of Alzheimer's disease” refers to the prophylactic treatment of AD; or delaying the onset or progression of AD. For example, the onset or progression of AD is delayed for at least 0.5, 1, 2, 3, 4, 5, 6, 7, 8, 9, or 10 years. In one embodiment, “prevention of Alzheimer's disease” refers to the prophylactic treatment of preclinical AD; or delaying the onset or progression of preclinical AD. In a further embodiment, the onset or progression of preclinical AD is delayed for at least 0.5, 1, 2, 3, 4, 5, 6, 7, 8, 9, or 10 years. In another embodiment, “prevention of Alzheimer's disease” refers to the prophylactic treatment of clinical AD; or delaying the onset or progression of clinical AD. In a further embodiment, the onset or progression of clinical AD is delayed for at least 0.5, 1, 2, 3, 4, 5, 6, 7, 8, 9, or 10 years.

As used herein, the term “clinical Alzheimer's disease” or “clinical AD” encompasses both Mild Cognitive Impairment (MCI) due to AD and dementia due to AD, unless the context makes clear that either only MCI due to AD or dementia due to AD is intended. The European Medicines Agency (EMA) in its ‘Draft guidelines on the clinical investigation of medicines for the treatment of AD and other dementias’ (EMA/Committee for Medicinal Products for Human Use (CHMP)/539931/2014) summarises the National Institute on Aging criteria for the diagnosis of MCI due to AD and AD dementia as set out below.

Diagnosis of MCI due to AD requires evidence of intra-individual decline, manifested by:
a) A change in cognition from previously attained levels, as noted by self- or informant report and/or the judgment of a clinician.
b) Impaired cognition in at least one domain (but not necessarily episodic memory) relative to age- and education-matched normative values; impairment in more than one cognitive domain is permissible.
c) Preserved independence in functional abilities, although the criteria also accept ‘mild problems’ in performing instrumental activities of daily living (IADL) even when this is only with assistance (i.e. rather than insisting on independence, the criteria allow for mild dependence due to functional loss).
d) No dementia, which nominally is a function of c (above).
e) A clinical presentation consistent with the phenotype of AD in the absence of other potentially dementing disorders. Increased diagnostic confidence may be suggested by
   1) Optimal: A positive Aβ biomarker and a positive degeneration biomarker
   2) Less optimal:
      i. A positive Aβ biomarker without a degeneration biomarker
      ii. A positive degeneration biomarker without testing for Aβ biomarkers

Diagnosis of AD dementia requires:
a) The presence of dementia, as determined by intra-individual decline in cognition and function.
b) Insidious onset and progressive cognitive decline.
c) Impairment in two or more cognitive domains; although an amnestic presentation is most common, the criteria allow for diagnosis based on nonamnestic presentations (for example impairment in executive function and visuospatial abilities).
d) Absence of prominent features associated with other dementing disorders.

Increased diagnostic confidence may be suggested by the biomarker algorithm discussed in the MCI due to AD section above.

As used herein, the term “preclinical Alzheimer's disease” or “preclinical AD” refers to the presence of in vivo molecular biomarkers of AD in the absence of clinical symptoms. The National Institute on Aging and Alzheimer's Association provide a scheme, shown in Table A below, which sets out the different stages of preclinical AD (Sperling et al., 2011).

TABLE A
Preclinical AD staging categories

| | | | Markers of | |
|---|---|---|---|---|
| | | | neuronal | Evidence |
| | | | injury | of subtle |
| | | Aβ (PET | (tau, FDG, | cognitive |
| Stage | Description | or CSF) | sMRI) | change |
| Stage 1 | Asymptomatic | Positive | Negative | Negative |
| | cerebral | | | |
| | amyloidosis | | | |
| Stage 2 | Asymptomatic | Positive | Positive | Negative |
| | amyloidosis + | | | |
| | “downstream” | | | |
| | neurodegeneration | | | |
| Stage 3 | Amyloidosis + | Positive | Positive | Positive |
| | neuronal injury + | | | |
| | subtle cognitive/ | | | |
| | behavioral decline | | | |

sMRI = structural magnetic resonance imaging

As used herein, the term “Cerebral Amyloid Angiopathy” or “CAA” refers to a disease characterised by the accumulation of β-amyloid (Aβ) proteins in the walls of cortical and leptomeningeal blood vessels. CAA is a common cause of vessel wall breakdown and vascular dysfunction in older adults, making it a major contributor to fatal or disabling intracerebral hemorrhages (ICH) as well as ischemic injury and dementia (Gurol M E et al., 2016). As used herein, the term “Cerebral Amyloid Angiopathy” or “CAA” encompasses both CAA-Type 1 and CAA-Type 2 unless the context makes clear that only CAA-Type 1 or CAA-Type 2 is intended.

As used herein, the term “CAA-Type 1” refers to capillary CAA (capCAA) characterised by Aβ protein depositions in the walls of cortical capillaries (Thal et al., 2002).

As used herein, the term “CAA-Type 2” refers to CAA characterised by Aβ protein depositions in the walls of leptomeningeal and cortical vessels, with the exception of cortical capillaries (Thal et al., 2002).

As used herein, the term “treatment of CAA” refers to the administration of the Compound of Formula 1, especially polymorph Form A to a patient in order to slow or arrest the development of CAA or at least one of the clinical symptoms of CAA, for example ICH, ischemic injury, or dementia. The development of CAA may be assessed by measuring the accumulation of Aβ in the walls of cortical (for example occipital cortex) and leptomeningeal blood vessels using a Positron Emission Tomography (PET) tracer, for example ¹⁸F-florbetapir (Gurol M E et al., 2016). Alternatively, the development of CAA may be assessed by monitoring cerebral microbleeds (CMB) as a haemorrhagic marker of CAA (Greenberg S M et al., 2014). Suitable techniques for the monitoring of CMB include, for example, magnetic resonance imaging (MRI) susceptibility-weighted imaging (SWI) and MRI T2*-weighted gradient-recalled echo imaging (GRE), and are described in Cheng A L et al., 2013. In addition, white matter hyperintensities (WMH) occur at much greater volume in patients diagnosed with CAA than in healthy aged individuals or in patients suffering from AD or mild cognitive impairment (MCI) (Greenberg S M et al., 2014). Therefore, CAA development may be monitored by the measurement of WMH volume using MRI (Chen Y W et al., 2006). It is expected that the “treatment of CAA” will have the resultant benefit of reducing the likelihood of a cerebral ischemic event in the patient undergoing treatment for CAA. Therefore, in one embodiment of the invention, the term “treatment of CAA” is equivalent to the term “treatment of intracerebral haemorrhage”. In another embodiment of the invention, the term “treatment of CAA” is equivalent to the term “treatment of CAA and/or intracerebral haemorrhage”. In a further embodiment of the invention, the term “treatment of CAA” is equivalent to the term “treatment of CAA and intracerebral haemorrhage associated therewith”.

As used herein, the term “prevention of CAA” refers to the prophylactic treatment of CAA; delaying the onset or progression of CAA; or delaying the onset or progression of at least one of the clinical symptoms of CAA. For example, the onset or progression of CAA is delayed for at least 0.5, 1, 2, 3, 4, 5, 6, 7, 8, 9, or 10 years. In one embodiment of the invention, the term “prevention of CAA” is equivalent to the term “prevention of intracerebral haemorrhage”. In another embodiment of the invention, the term “prevention of CAA” is equivalent to the term “prevention of CAA and/or intracerebral haemorrhage”. In a further embodiment of the invention, the term “prevention of CAA” is equivalent to the term “prevention of CAA and intracerebral haemorrhage associated therewith”.

As used herein, the term “a genetic predisposition for the development of CAA” includes, but is not limited to situations where the genetic predisposition is due to: Down's syndrome; a mutation in the gene for amyloid precursor protein or presenilin-1; or the presence of one or two copies of the ApoE4 allele.

As used herein, the term “patient” refers to a human subject.

The term “a therapeutically effective amount” refers to an amount of Compound 1 that will elicit inhibition of BACE-1 in a patient as evidenced by a reduction in CSF or plasma Aβ 1-40 levels relative to an initial baseline value. Aβ 1-40 levels may be measured using standard immunoassay techniques, for example Meso Scale Discovery (MSD) 96-well MULTI-ARRAY human/rodent (4G8) Aβ40 Ultrasensitive Assay (# K110FTE-3, Meso Scale Discovery, Gaithersburg, USA).

List of Abbreviations

| Abbreviation | Description |
|---|---|
| ACN | acetonitrile |
| APP | amyloid precursor protein |
| Aβ | beta-amyloid peptide |
| aq. | aqueous |
| Aβ40 | beta-amyloid peptide 40 |
| BACE-1 | beta site APP cleaving enzyme-1 |
| BACE | beta site APP cleaving enzyme |
| Boc₂O | di-tert-butyl dicarbonate |
| b.p. | boiling point |
| BuLi or nBuLi | n-butyllithium |
| C | concentration |
| CI | confidence interval |
| CDCl₃ | deuterated chloroform |
| conc. | concentrated |
| CSF | cerebrospinal fluid |
| Cu₂O | copper(I) oxide |
| d | day |
| δ | chemical shift in ppm |
| DCM | dichloromethane |
| DMF | N,N-dimethylformamide |
| DMSO | dimethylsulfoxide |
| DSC | differential scanning calorimetry |
| EDC | 1-(3-dimethylaminopropyl)-3- |
| | ethylcarbodiimide hydrochloride |
| ESI | electrospray ionisation |
| EtOAc | ethyl acetate |
| g | gram |
| h, hr | hour(s) |
| HCl | hydrochloric acid |
| Hex | hexane |
| HOAt | 1-hydroxy-7-azabenzotriazole |
| HPLC, LC | high-performance liquid chromatography, |
| | liquid chromatography |
| IPAc | isopropyl acetate |
| K₂CO₃ | potassium carbonate |
| kJ | kilojoule |
| kg | kilogram |
| KOtBu | potassium tert-butoxide |
| kV | kilovolt |
| LC-MS/MS | tandem mass spectrometry |
| mA | milliampere |
| mDSC | modulated differential scanning calorimetry |
| MeOH | methanol |
| MHz | megahertz |
| min | minute |
| ml/mL | milliliter |
| mm | millimeter |
| μl | microliter |
| μm | micrometer |
| μM | micromolar |
| μmol | micromoles |
| min | minute(s) |
| mmol | millimoles |
| MS | mass spectrometry |
| NaHCO₃ | sodium bicarbonate |
| Na₂SO₄ | sodium sulfate |
| NEt₃ | triethylamine |
| nm | nanometer |
| nM | nanomolar |
| NMR | nuclear magnetic resonance spectrometry |
| PI | pharmaceutical intermediate |
| PK | pharmacokinetic |
| ppm | parts per million |
| q.d. or QD | quaque die |
| Rf | retention factor |
| RH | relative humidity |
| rpm | revolutions per minute |
| Rt | retention time (min) |
| RT, rt | room temperature |
| s | second |
| SD | single dose |
| T | time |
| TBME | tert-butyl methyl ether |
| TFA | trifluoroacetic acid |
| TGA | thermogravimetric analysis |
| THF | tetrahydrofuran |
| TLC | thin layer chromatography |
| UPLC | ultra performance liquid chromatography |
| v/v | by volume |
| w/w | by weight |
| WL | copper Kα radiation wavelength (λ_{Cu} = 1.5406 Å) |
| wt | weight ratio based on the quantity of starting material |
| XRPD | x-ray powder diffraction |

### EXAMPLES

The following Examples illustrate various aspects of the invention. Examples 1 and 2 show how Compound 1 may be prepared and how it may be crystallised to produce Form A. Examples 3 and 4 describe the XRPD and DSC analysis of Form A. Example 5 describes the micronization procedure of Form A and the corresponding XRPD data. Examples 6 and 7 describe formulations comprising Form A and their method of manufacture. Examples 8, 9 and 10 describe the process of making Form B, and the XRPD and DSC analysis of Form B. Examples 11, 12 and 13 describe the process of making the hydrate Form H_{A}, and the DSC and XRPD analysis of the hydrate Form H_{A}. Examples 14 and 15 describe the process for making the amorphous form and the mDSC analysis. Example 16 shows the stability data of Form A. Example 17 describes the in human study of pharmacokinetics of free base Compound 1 when given alone and in combination with the strong CYP3A4 inhibitor itraconazole or the strong CYP3A4 inducer rifampicin.

### Example 1: Preparation of Compound 1

The preparation of Compound 1 is described in WO 2012/095469 A1 (Example 34). Compound 1 may also be prepared as described below.

### NMR Methodology

Proton spectra are recorded on a Bruker 400 MHz ultrashield spectrometer unless otherwise noted. Chemical shifts are reported in ppm relative to methanol (δ 3.31), dimethyl sulfoxide (δ 2.50), or chloroform (δ 7.29). A small amount of the dry sample (2-5 mg) is dissolved in an appropriate deuterated solvent (0.7 mL). The shimming is automated and the spectra obtained in accordance with procedures well known to the person of ordinary skill in the art.

### XRPD Method for Form A:

X-ray powder diffraction (XRPD) analysis was performed using a Bruker D8 Advance x-ray diffractometer in reflection geometry. Measurements were taken at about 30 kV and 40 mA under the following conditions:

TABLE 1a

| | |
|---|---|
| Scan rate (continuous scan): | 3 s/step |
| Step size: | 0.017° (2-theta) |
| Soller slit: | 2.5° |
| Slits (from left to right): | V12 (variable) |

The X-ray diffraction pattern was recorded between 2° and 40° (2-theta) with CuK_{α} radiation for identification of the whole pattern.

### XRPD Method for Form B, Hydrate H_{A}, Amorphous Form:

X-ray powder diffraction (XRPD) analysis was performed using a Bruker D8 Advance x-ray diffractometer in reflection geometry. Measurements were taken at about 40 kV and 40 mA under the following conditions:

TABLE 1b

| | |
|---|---|
| Scan rate (continuous scan): | 39 s/step |
| Step size: | 0.016° (2-theta) |
| Soller slit: | 2.5° |
| Slits: | Primary: fixed illuminated sample size |
| | 10 mm, secondary slit: 5 mm |

The X-ray diffraction pattern was recorded between 2° and 45° (2-theta) with CuK_{α} radiation for identification of the whole pattern. All 2-theta (29) values are within +/−0.2°.

### General Chromatography Information

### HPLC Method H1 (Rt_{H1}):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 30-100% B in 3.25 min, flow=0.7 ml/min

### LCMS Method H2 (Rt_{H2}):

HPLC-column dimensions: 3.0×30 mm
HPLC-column type: Zorbax SB-C18, 1.8 μm
HPLC-eluent: A) water+0.05 Vol.-% TFA, B) ACN+0.05 Vol.-% TFA
HPLC-gradient: 10-100% B in 3.25 min, flow=0.7 ml/min

### UPLCMS Method H3 (Rt_{H3}):

HPLC-column dimensions: 2.1×50 mm
HPLC-column type: Acquity UPLC HSS T3, 1.8 μm
HPLC-eluent: A) water+0.05 Vol.-% formic acid+3.75 mM ammonium acetate B) ACN+0.04 Vol.-% formic acid
HPLC-gradient: 2-98% B in 1.4 min, 98% B 0.75 min, flow=1.2 ml/min
HPLC-column temperature: 50° C.

### LCMS Method H4 (Rtₕ₄):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 70-100% B in 3.25 min, flow=0.7 ml/min

### LCMS Method H5 (Rtₕ₅):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 80-100% B in 3.25 min, flow=0.7 ml/min

### LCMS Method H6 (Rt_{H6}):

HPLC-column dimensions: 3.0×30 mm
HPLC-column type: Zorbax SB-C18, 1.8 μm
HPLC-eluent: A) water+0.05 Vol.-% TFA; B) ACN+0.05 Vol.-% TFA
HPLC-gradient: 40-100% B in 3.25 min, flow=0.7 ml/min

### a) 2-Bromo-5-fluoro-4-triethylsilanyl-pyridine

A solution of diisopropylamine (25.3 g, 250 mmol) in 370 ml THF was cooled with a dry-ice acetone bath at −75° C. BuLi (100 ml, 250 mmol, 2.5 M in hexanes) was added dropwise while maintaining the temperature below −50° C. After the temperature of the mixture had reached −75° C. again, a solution of 2-bromo-5-fluoropyridine (36.7 g, 208 mmol) in 45 ml THF was added dropwise. The mixture was stirred for 1 h at −75° C. Triethylchlorosilane (39.2 g, 260 mmol) was added quickly. The temperature stayed below −50° C. The cooling bath was removed and the reaction mixture was allowed to warm to −15° C., poured onto aq. NH₄Cl (10%). TBME was added and the layers were separated. The organic layer was washed with brine, dried with MgSO₄.H₂O, filtered and evaporated to give a brown liquid which was distilled at 0.5 mm Hg to yield the title compound as a slightly yellow liquid (b.p. 105-111° C.). HPLC: Rt_{H4}=2.284 min; ESIMS: 290, 292 [(M+H)⁺, 1Br]; ¹H-NMR (400 MHz, CDCl₃): 8.14 (s, 1H), 7.40 (d, 1H), 1.00-0.82 (m, 15H).

### b) 1-(6-Bromo-3-fluoro-4-triethylsilanyl-pyridin-2-yl)-ethanone

A solution of diisopropylamine (25.4 g, 250 mmol) in 500 ml THF was cooled to −75° C. BuLi (100 ml, 250 mmol, 2.5 M in hexanes) was added dropwise while maintaining the temperature below −50° C. After the reaction temperature had reached −75° C. again, a solution of 2-bromo-5-fluoro-4-triethylsilanyl-pyridine (56.04 g, 193 mmol) in 60 ml THF was added dropwise. The mixture was stirred in a dry ice bath for 70 minutes. N,N-dimethylacetamide (21.87 g, 250 mmol) was added quickly, the reaction temperature rose to −57° C. The reaction mixture was stirred in a dry ice bath for 15 min and then allowed to warm to −40° C. It was poured on a mixture of 2M aq. HCl (250 ml, 500 mmol), 250 ml water and 100 ml brine. The mixture was extracted with TBME, washed with brine, dried over MgSO₄.H₂O, filtered and evaporated to give a yellow oil which was purified on a silica gel column by eluting with hexane/0-5% TBME to yield 58.5 g of the title compound as a yellow liquid. TLC (Hex/TBME 99/1): R_{f}=0.25; HPLC: Rt_{H4}=1.921 min; ESIMS: 332, 334 [(M+H)⁺, 1Br]; ¹H-NMR (400 MHz, CDCl₃): 7.57 (d, 1H), 2.68 (s, 3H), 1.00-0.84 (m, 15H).

### c) (S)-2-(6-Bromo-3-fluoro-4-triethylsilanyl-pyridin-2-yl)-2-trimethylsilanyloxy-propionitrile

At first, the catalyst solution was prepared by dissolving water (54 mg, 3.00 mmol) in 100 ml dry DCM (≤0.001% water). This wet DCM (44 ml, 1.32 mmol water content) was added to a well stirred solution of titanium(IV) butoxide (500 mg, 1.47 mmol) in 20 ml dry DCM. The resulting clear solution was refluxed for 1 h. This solution was then cooled to rt and 2,4-di-tert-butyl-6-{[(E)-(S)-1-hydroxymethyl-2-methyl-propylimino]-methyl}-phenol [CAS 155052-31-6] (469 mg, 1.47 mmol) was added. The resulting yellow solution was stirred at rt for 1 h. This catalyst solution (0.023 M, 46.6 ml, 1.07 mmol) was added to a solution of 1-(6-bromo-3-fluoro-4-triethylsilanyl-pyridin-2-yl)-ethanone (35.53 g, 107 mmol) and trimethylsilyl cyanide (12.73 g, 128 mmol) in 223 ml dry DCM. The mixture was stirred for 2 days and evaporated to give 47 g of the crude title compound as an orange oil. HPLC: Rt_{H5}=2.773 min; ESIMS: 431, 433 [(M+H)⁺, 1Br]; ¹H-NMR (400 MHz, CDCl₃): 7.46 (d, 1H), 2.04 (s, 3H), 1.00 (t, 9H), 1.03-0.87 (m, 15H), 0.20 (s, 9H).

### d) (R)-1-Amino-2-(6-bromo-3-fluoro-4-triethylsilanyl-pyridin-2-yl)-propan-2-ol hydrochloride

Borane dimethyl sulfide complex (16.55 g, 218 mmol) was added to a solution of crude (S)-2-(6-bromo-3-fluoro-4-triethylsilanyl-pyridin-2-yl)-2-trimethylsilanyloxy-propionitrile (47 g, 109 mmol) in 470 ml THF. The mixture was refluxed for 2 h. The heating bath was removed and the reaction mixture was quenched by careful and dropwise addition of MeOH. After the evolution of gas had ceased, aq. 6M HCl (23.6 ml, 142 mmol) was added slowly. The resulting solution was evaporated and the residue was dissolved in MeOH and evaporated (twice) to yield 44.5 g of a yellow foam, pure enough for further reactions. HPLC: Rt_{H1}=2.617 min; ESIMS: 363, 365 [(M+H)⁺, 1Br]; ¹H-NMR (400 MHz, CDCl₃): 7.93 (s, br, 3H), 7.53 (d, 1H), 6.11 (s, br, 1H), 3.36-3.27 (m, 1H), 3.18-3.09 (m, 1H), 1.53 (s, 3H), 0.99-0.81 (m, 15H).

### e) (R)—N-(2-(6-bromo-3-fluoro-4-(triethylsilyl)pyridin-2-yl)-2-hydroxypropyl)-4-nitrobenzenesulfonamide

To a solution of crude (R)-1-amino-2-(6-bromo-3-fluoro-4-triethylsilanyl-pyridin-2-yl)-propan-2-01 hydrochloride (43.5 g, 109 mmol) in 335 ml THF was added a solution of NaHCO₃ (21.02 g, 250 mmol) in 500 ml water. The mixture was cooled to 0-5° C. and a solution of 4-nitrobenzenesulfonyl chloride (26.5 g, 120 mmol) in 100 ml THF was added in a dropwise. The resulting emulsion was stirred overnight while allowing the temperature to reach rt. The mixture was extracted with TBME. The organic layer was dried with MgSO₄.H₂O, filtered and evaporated to give an orange resin which was purified on a silca gel column by eluting with Hexanes/10-20% EtOAc to yield 37.56 g of the title compound as a yellow resin. TLC (Hex/EtOAc 3/1): R_{f}=0.34; HPLC: Rt_{H4}=1.678 min; ESIMS: 548, 550 [(M+H)⁺, 1Br]; ¹H-NMR (400 MHz, DMSO-d₆): 8.40 (d, 2H), 8.06 (t, 1H), 7.97 (d, 2H), 7.45 (d, 1H), 5.42 (s, 1H), 3.23 (d, 2H), 1.44 (s, 3H) 0.97-0.81 (m, 15H); Chiral HPLC (Chiralpak AD-H 1213, UV 210 nm): 90% ee.

### f) 6-Bromo-3-fluoro-2-[(S)-2-methyl-1-(4-nitro-benzenesulfonyl)-aziridin-2-yl]-4-triethylsilanyl-pyridine

A solution of triphenylphosphine (21.55 g, 82 mmol) and (R)—N-(2-(6-bromo-3-fluoro-4-(triethylsilyl)pyridin-2-yl)-2-hydroxypropyl)-4-nitrobenzenesulfonamide (37.56 g, 69 mmol) in 510 ml THF was cooled to 4° C. A solution of diethyl azodicarboxylate in toluene (40% by weight, 38.8 g, 89 mmol) was added in a dropwise while maintaining the temperature below 10° C. The cooling bath was removed and the reaction mixture was stirred at rt for 1 h. The reaction mixture was diluted with approx. 1000 ml toluene and THF was removed under reduced pressure. The resulting toluene solution of crude product was pre-purified on a silca gel column by eluting with hexanes/5-17% EtOAc. Purest fractions were combined, evaporated and crystallized from TBME/hexane to yield 29.2 g of the title compound as white crystals. HPLC: Rt_{H4}=2.546 min; ESIMS: 530, 532 [(M+H)⁺, 1Br]; ¹H-NMR (400 MHz, CDCl₃): 8.40 (d, 2H), 8.19 (d, 2H), 7.39 (d, 1H), 3.14 (s, 1H), 3.02 (s, 1H), 2.01 (s, 3H) 1.03-0.83 (m, 15H); α[D] −35.7° (c=0.97, DCM).

### g) 6-Bromo-3-fluoro-2-[(S)-2-methyl-1-(4-nitro-benzenesulfonyl)-aziridin-2-yl]-pyridine

Potassium fluoride (1.1 g, 18.85 mmol) was added to a solution of 6-bromo-3-fluoro-2-[(S)-2-methyl-1-(4-nitro-benzenesulfonyl)-aziridin-2-yl]-4-triethylsilanyl-pyridine (5 g, 9.43 mmol) and AcOH (1.13 g, 9.43 mmol) in 25 ml THF. DMF (35 ml) was added and the suspension was stirred for 1 h at rt. The reaction mixture was poured onto a mixture of sat. aq. NaHCO₃ and TBME. The layers were separated and washed with brine and TBME. The combined organic layers were dried over MgSO₄.H₂O, filtered and evaporated to give a yellow oil which was crystallized from TBME/hexane to yield 3.45 g of the title compound as white crystals. HPLC: Rt_{H6}=2.612 min; ESIMS: 416, 418 [(M+H)⁺, 1Br]; ¹H-NMR (400 MHz, CDCl₃): 8.41 (d, 2H), 8.19 (d, 2H), 7.48 (dd, 1H), 7.35 (t, 1H), 3.14 (s, 1H), 3.03 (s, 1H), 2.04 (s, 3H); α[D]−35.7° (c=0.89, DCM).

### h) (R)-2-[(R)-2-(6-Bromo-3-fluoro-pyridin-2-yl)-2-(4-nitro-benzenesulfonylamino)-propoxy]-3,3,3-trifluoro-2-methyl-propionic acid ethyl ester

A solution of (R)-3,3,3-trifluoro-2-hydroxy-2-methyl-propionic acid ethyl ester (11.93 g, 64.1 mmol) in DMF (158 ml) was evacuated/flushed with nitrogen twice. A solution of KOtBu (6.21 g, 55.5 mmol) in DMF (17 ml) was added dropwise while maintaining a reaction temperature of ca 25° C. using cooling with a water bath. After 15 min solid 6-bromo-3-fluoro-2-[(S)-2-methyl-1-(4-nitro-benzenesulfonyl)-aziridin-2-yl]-pyridine (17.78 g, 42.7 mmol) was added and stirring was continued for 3 h. The reaction mixture was poured onto a mixture of 1M HCl (56 ml), brine and TBME. The layers were separated, washed with brine and TBME. The combined organic layers were dried over MgSO₄.H₂O, filtered and evaporated. The crude reaction product was purified via chromatography on silica gel (hexanes/25-33% TBME) to yield 16.93 g of the title compound as a yellow resin that was contaminated with an isomeric side-product (ratio 70:30 by ¹H-NMR).

HPLC: Rt_{H6}=2.380 min; ESIMS: 602, 604 [(M+H)⁺, 1Br]; ¹H-NMR (400 MHz, CDCl₃): 8.32 (d, 2H), 8.07 (d, 2H), 7.46-7.41 (m, 1H), 7.30-7.23 (m, 1H), 6.92 (s, 1H), 3.39-4.30 (m, 2H), 3.95 (d, 1H), 3.84 (d, 1H), 1.68 (s, 3H), 1.56 (s, 3H), 1.40-1.34 (m, 3H)+isomeric side-product.

### i) (R)-2-[(R)-2-(6-Bromo-3-fluoro-pyridin-2-yl)-2-(4-nitro-benzenesulfonylamino)-propoxy]-3,3,3-trifluoro-2-methyl-propionamide

A solution of (R)-2-[(R)-2-(6-bromo-3-fluoro-pyridin-2-yl)-2-(4-nitro-benzenesulfonylamino)-propoxy]-3,3,3-trifluoro-2-methyl-propionic acid ethyl ester (16.93 g, 28.1 mmol) in a NH₃/MeOH (7M, 482 ml) was stirred at 50° C. in a sealed vessel for 26 h. The reaction mixture was evaporated and the residue was crystallized from DCM to yield 9.11 g of the title compound as colorless crystals.

HPLC: Rt_{H6}=2.422 min; ESIMS: 573, 575 [(M+H)⁺, 1Br]; ¹H-NMR (400 MHz, CDCl₃): 8.33 (d, 2H), 8.06 (d, 2H), 7.42 (dd, 1H), 7.30-7.26 (m, 1H), 7.17 (s, br, 1H), 6.41 (s, 1H), 5.57 (s, br, 1H), 4.15 (m, 2H), 1.68 (s, 3H), 1.65 (s, 3H).

### j) N—[(R)-1-(6-Bromo-3-fluoro-pyridin-2-yl)-2-((R)-1-cyano-2,2,2-trifluoro-1-methyl-ethoxy)-1-methyl-ethyl]-4-nitro-benzenesulfonamide

A suspension of (R)-2-[(R)-2-(6-bromo-3-fluoro-pyridin-2-yl)-2-(4-nitro-benzenesulfonylamino)-propoxy]-3,3,3-trifluoro-2-methyl-propionamide (8.43 g, 14.70 mmol) and triethylamine (5.12 ml, 36.8 mmol) in 85 ml DCM was cooled to 0-5° C. Trifluoroacetic anhydride (2.49 ml, 17.64 mmol) was added dropwise over 30 min. Additional triethylamine (1.54 ml, 11.07 mmol) and trifluoroacetic anhydride (0.75 ml, 5.29 mmol) were added to complete the reaction. The reaction mixture was quenched by addition of 14 ml aqueous ammonia (25%) and 14 ml water. The emulsion was stirred for 15 min, more water and DCM were added and the layers were separated. The organic layer was dried with MgSO₄H₂O, filtered and evaporated. Purification by column chromatography on a silica gel (hexanes/10-25% EtOAc) gave 8.09 g of the title compound as a yellow resin.

HPLC: Rt_{H6}=3.120 min; ESIMS: 555, 557 [(M+H)⁺, 1Br]; ¹H-NMR (400 MHz, CDCl₃): 8.35 (d, 2H), 8.11 (d, 2H), 7.50 (dd, 1H), 7.32 (dd, 1H), 6.78 (s, 1H), 4.39 (d 1H), 4.22 (d, 1H), 1.68 (s, 6H).

### k) (2R,5R)-5-(6-Bromo-3-fluoro-pyridin-2-yl)-2,5-dimethyl-2-trifluoromethyl-5,6-dihydro-2H-[1,4]oxazin-3-ylamine

A solution of N—[(R)-1-(6-bromo-3-fluoro-pyridin-2-yl)-2-((R)-1-cyano-2,2,2-trifluoro-1-methyl-ethoxy)-1-methyl-ethyl]-4-nitro-benzenesulfonamide (9.18 g, 16.53 mmol) and N-acetylcysteine (5.40 g, 33.10 mmol) in 92 ml ethanol was evacuated and flushed with nitrogen. K₂CO₃ (4.57 g, 33.1 mmol) was added and the mixture was stirred at 80° C. for 3 days. The reaction mixture was concentrated in vacuo to about ¼ of the original volume and partitioned between water and TBME. The organic layer was washed with 10% aq. K₂CO₃ solution, dried over Na₂SO₄, filtered and evaporated to give a yellow oil. Column chromatography on silica (hexanes/14-50% (EtOAc:MeOH 95:5)) gave 4.55 g of the title compound as an off-white solid.

HPLC: Rt_{H2}=2.741 min; ESIMS: 370, 372 [(M+H)⁺, 1Br]; ¹H-NMR (400 MHz, DMSO-d₆): 7.71-7.62 (m, 2H), 5.97 (s, br, 2H), 4.02 (d 1H), 3.70 (d, 1H), 1.51 (s, 3H), 1.47 (s, 3H).

### l) (2R,5R)-5-(6-Amino-3-fluoro-pyridin-2-yl)-2,5-dimethyl-2-trifluoromethyl-5,6-dihydro-2H-[1,4]oxazin-3-yl amine

A glass/stainless steel autoclave was purged with nitrogen, Cu₂O (0.464 g, 3.24 mmol), ammonia (101 ml, 25%, aq., 648 mmol, 30 equivalents) and (2R,5R)-5-(6-Bromo-3-fluoro-pyridin-2-yl)-2,5-dimethyl-2-trifluoromethyl-5,6-dihydro-2H-[1,4]oxazin-3-ylamine (8 g, 21.6 mmol) in ethylene glycol (130 ml) was added. The autoclave was closed and the suspension heated up to 60° C. and the solution was stirred for about 48 hours (max. pressure 0.7 bar, inside temperature 59-60° C.). The reaction mixture was diluted with ethyl acetate and water. The organic phase was washed with water and 4 times with 12% aq. ammonia and finally with brine, dried over sodium sulfate, filtered and evaporated. The crude product (7 g, containing some ethylen glycol, quantitative yield) was used in the next step without further purification.

HPLC: Rt_{H3}=0.60 min; ESIMS: 307 [(M+H)⁺].

### m) [(2R,5R)-5-(6-Amino-3-fluoro-pyridin-2-yl)-2,5-dimethyl-2-trifluoromethyl-5,6-dihydro-2H-[1,4]oxazin-3-yl]-carbamic acid tert-butyl ester

A solution of (2R,5R)-5-(6-amino-3-fluoro-pyridin-2-yl)-2,5-dimethyl-2-trifluoromethyl-5,6-dihydro-2H-[1,4]oxazin-3-yl amine (6.62 g, 21.6 mmol), Boc₂O (4.72 g, 21.6 mmol) and Hunig's base (5.66 ml, 32.4 mmol) in dichloromethane (185 ml) was stirred at rt for 18 hours. The reaction mixture was washed with sat. aq. NaHCO₃ and brine. The aqueous layers were back extracted with dichloromethane and the combined organic layers were dried over sodium sulfate, filtered and evaporated to give a light green solid (14 g). The crude product was chromatographed over silicagel (cyclohexane:ethyl acetate 95:5 to 60:40) to afford 7.68 g of the title compound.

TLC (cyclohexane:ethyl acetate 3:1): R_{f}=0.21; HPLC: Rt_{H3}=1.14 min; ESIMS: 408 [(M+H)⁺]; ¹H-NMR (400 MHz, CDCl3): 11.47 (br. s, 1H), 7.23 (dd, J=10.42, 8.78 Hz, 1H), 6.45 (dd, J=8.78, 2.64 Hz, 1H), 4.50 (br. s, 2H), 4.32 (d, J=2.38 Hz, 1H), 4.10 (d, J=11.80 Hz, 1H), 1.69 (s, 3H, CH3), 1.65 (s, 3H, CH3), 1.55 (s, 9H).

### n) ((2R,5R)-5-{6-[(3-Chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester

A mixture of [(2R,5R)-5-(6-amino-3-fluoro-pyridin-2-yl)-2,5-dimethyl-2-trifluoromethyl-5,6-dihydro-2H-[1,4]oxazin-3-yl]-carbamic acid tert-butyl ester (3.3 g, 8.12 mmol), 3-chloro-5-trifluoromethylpicolinic acid (2.2 g, 9.74 mmol), HOAt (1.99 g, 14.62 mmol) and EDC hydrochloride (2.33 g, 12.18 mmol) was stirred in DMF (81 ml) at rt for 48 hours. The reaction mixture was diluted with ethyl acetate and washed with water and brine, dried over sodium sulfate, filtered and evaporated. The crude product (12 g) was chromatographed over silicagel (cyclohexane to cyclohexane:ethyl acetate 1:1) to yield 5.2 g of the title compound.

TLC (silica, cyclohexane:ethyl acetate 3:1): R_{f}=0.47; HPLC: Rt_{H3}=1.40 min; ESIMS: 615, 616 [(M+H)⁺, 1Cl]; ¹H-NMR (400 MHz, CDCl₃): 11.68 (s, 1H), 10.41 (s, 1H), 8.81 (dd, J=1.82, 0.69 Hz, 1H), 8.45 (dd, J=8.91, 3.14 Hz, 1H), 8.19 (dd, J=1.88, 0.63 Hz, 1H), 7.59 (dd, J=9.79, 9.16 Hz, 1H), 4.38 (d, J=2.13 Hz, 1H), 4.18 (d, J=11.80 Hz, 1H), 1.75 (s, 3H), 1.62 (s, 3H), 1.60 (s, 9H).

### o) 3-Chloro-5-trifluoromethyl-pyridine-2-carboxylic acid [6-((3R,6R)-5-amino-3,6-dimethyl-6-trifluoromethyl-3,6-dihydro-2H-[1,4]oxazin-3-yl)-5-fluoro-pyridin-2-yl]-amide

A mixture of ((2R,5R)-5-{6-[3-chloro-5-trifluoromethyl-pyridine-2-carbonyl)-amino]-3-fluoro-pyridin-2-yl}-2,5-dimethyl-2-trifluoromethyl-5,6-dihydro-2H-[1,4]oxazin-3-yl)-carbamic acid tert-butyl ester (4.99 g, 8.13 mmol) and TFA (6.26 ml, 81 mmol) in dichloromethane (81 ml) was stirred at rt for 18 hours. The solvent was evaporated and the residue diluted with a suitable organic solvent, such as ethyl acetate and aq. ammonia. Ice was added and the organic phase was washed with water and brine, dried over sodium sulfate, filtered and evaporated to yield 3.78 g of the title compound.

HPLC: Rt_{H3}=0.87 min; ESIMS: 514, 516 [(M+H)⁺, 1Cl]; ¹H-NMR (400 MHz, DMSO-d₆): δ 11.11 (s, 1H), 9.06 (s, 1H), 8.69 (s, 1H), 8.13 (dd, J=8.8, 2.6 Hz, 1H), 7.80-7.68 (m, 1H), 5.88 (br. s, 2H), 4.12 (d, J=11.5 Hz, 1H), 3.72 (d, J=11.4 Hz, 1H), 1.51 (s, 3H), 1.49 (s, 3H).

### Example 2: Crystallisation Procedure Form A

1 wt of Compound 1, obtained by the procedure of Example 1, was dissolved in 5.11 wt of IPAc at 70-80° C. The solution was filtered (filter <2 μm) and then 1.52 wt of n-heptane added. The solution was cooled to 55° C., and seeded with 0.5% w/w of Form A. The suspension was held at 55° C. for 30-60 mins and then cooled to 35° C. over 2 hours. The suspension was aged for 1 hour and then 8.2 wt of n-heptane were added over 3 hours. The suspension was aged for 1 hour and then cooled to 0-5° C. over 2 hours and aged for at least 2 hours. The suspension was filtered under vacuum, and the cake washed with 10/90 w/w isopropyl acetate/n-heptane. The cake was dried under vacuum at 40-45° C. until dry, to produce Form A.

### Example 3: XRPD Analysis of Form A

Crystalline Form A was analysed by XRPD and the ten most characteristic peaks are shown in Table 2 (see also FIG. 1).

TABLE 2

| 2-theta in degrees | relative intensity in % |
|---|---|
| 10.68 | 67.4 |
| 14.84 | 100.0 |
| 18.66 | 23.5 |
| 19.52 | 46.6 |
| 21.38 | 71.4 |
| 21.68 | 19.9 |
| 25.52 | 5.4 |
| 29.86 | 6.8 |
| 35.04 | 6.0 |
| 37.83 | 4.5 |

### Example 4: DSC Analysis of Form A

Crystalline Form A was analysed by differential scanning calorimetry (DSC) using a Discovery Diffraction Scanning Calorimeter from TA instruments and found to have an onset of melting at about 171° C., see FIG. 2. Heating rate was performed at 10° C. per minute.

### Example 5: Micronisation Procedure and XRPD for Micronized Form A

Crystalline Form A was micronised according to the following method:

A spiral jet-milling instrument was used with a ring of 50 mm diameter. The carrier gas was nitrogen and the energy was targeted at 1800 kJ/kg (cumulative parameter considering injector and grinding nozzle number and diameter, injector and grinding nozzle pressure, and feed rate according to Midoux et al., Powder Technol. 104 (1999) 113-120).

Micronised Crystalline Form A was analysed by XRPD and the ten most characteristic peaks are shown in Table 3 (see also FIG. 3).

TABLE 3

| 2-theta in degrees | relative intensity in % |
|---|---|
| 10.56 | 49.8 |
| 12.09 | 33.1 |
| 14.72 | 52.8 |
| 15.93 | 83.5 |
| 18.53 | 90.3 |
| 19.37 | 39.0 |
| 21.18 | 76.1 |
| 23.97 | 100.0 |
| 24.65 | 72.0 |
| 29.65 | 76.1 |

X-ray powder diffraction (XRPD) analysis was performed using a Bruker D8 Advance x-ray diffractometer in reflection geometry. Measurements were taken at about 30 kV and 40 mA under the conditions shown in Table 4.

TABLE 4

| | |
|---|---|
| Scan rate (continuous scan): | 3 s/step |
| Step size: | 0.017° (2-theta) |
| Soller slit: | 2.5° |
| Slits (from left to right): | V12 (variable) |

The X-ray diffraction pattern was recorded between 2° and 40° (2-theta) with CuK_{α} radiation for identification of the whole pattern.

### Example 6: Pharmaceutical Composition Comprising Form A—Formulation ‘A’

Form A was formulated as 1, 10, 25, and 75 mg dose strength hard gelatin capsules (for example Capsugel, size 3) comprising the ingredients shown in Table 5 (Formulation A). Batch manufacturing was carried out as described below and in Table 6.

TABLE 5
Composition of 1 mg, 10 mg, 25 mg and 75 mg Form A hard gelatin
capsule (Formulation A)

| | Formulation A amount | | | |
|---|---|---|---|---|
| | per capsule (% w/w) | | | |
| | 1 mg | 10 mg | 25 mg | 75 mg |
| Drug load | 0.6% | 5.9% | 14.7% | 44.1% |
| Capsule fill ingredient | | | | |
| Form A | 0.6 | 5.9 | 14.7 | 44.1 |
| Mannitol | 67.2 | 63.37 | 56.91 | 35.14 |
| Pregelatinized starch | 21.77 | 20.29 | 17.94 | 10.29 |
| Low substituted hydroxypropyl | 5.17 | 5.17 | 5.18 | 5.17 |
| cellulose | | | | |
| hydroxypropyl cellulose | 3.39 | 3.39 | 3.39 | 3.39 |
| Talc | 0.47 | 0.47 | 0.47 | 0.47 |
| Sodium stearyl fumarate | 1.41 | 1.41 | 1.41 | 1.41 |
| Weight capsule fill mix (mg) | 170.0 | 170.0 | 170.0 | 170.0 |

TABLE 6
Manufacturing of 1 mg, 10 mg, 25 mg and 75 mg hard gelatin capsules
of Form A (Formulation A)

| | Amount per batch (kg) | | | |
|---|---|---|---|---|
| | 1 mg | 10 mg | 25 mg | 75 mg³ |
| | 7500 | 16,000 | 35,000 | 7,100 |
| Batch size | units | units | units | units |
| Drug load | 0.6% | 5.9% | 14.7% | 44.1% |
| Capsule fill ingredient | | | | |
| Form A¹ | 0.0075 | 0.1600 | 0.875 | 0.5325 |
| Mannitol | 0.8568 | 1.7238 | 3.386 | 0.4242 |
| Pregelatinised starch | 0.2775 | 0.5520 | 1.068 | 0.1243 |
| Low-substituted Hydroxypropyl | 0.0660 | 0.1408 | 0.308 | 0.0625 |
| cellulose | | | | |
| Hydroxypropylcellulose | 0.0432 | 0.0922 | 0.202 | 0.0409 |
| Sodium stearyl fumarate | 0.0180 | 0.0384 | 0.084 | 0.0170 |
| Talc | 0.0060 | 0.0128 | 0.028 | 0.0057 |
| Purified water² | q.s. | q.s. | q.s. | q.s. |
| Weight capsule fill mix | 1.2750 | 2.7200 | 5.950 | 1.2071 |
| Empty capsule shell | | | | |
| Capsule shell, size 3 (theoretical | 0.3600 | 0.7680 | 1.680 | 0.3408 |
| weight) | | | | |
| Total batch weight | 1.6350 | 3.4880 | 7.630 | 1.5479 |

¹Corresponding to a corrected drug substance content (= cc) of 100%. A compensation of drug substance is performed if the corrected drug substance content is ≤99.5%. The difference in weight is adjusted with Mannitol.
²Removed during processing
³During granulation of the 75 mg strength formulation, it was observed that the granulation process was inadequate. This is likely attributed to the high drug load of 44% w/w in this composition. Therefore, for reliable granulation process, an upper limit to the drug load of, for example, 35% should be maintained.

Other batch sizes may be prepared depending on supply requirements and/or available equipment chain. The weight of individual components for other batch sizes corresponds proportionally to the stated composition.

### Description of Manufacturing Process of Form A Formulation A: 1 mg and 10 mg Hard Gelatin Capsules

1. Blend Form A drug substance and portion of mannitol.
2. Sieve the mixture of step 1.
3. Blend the mixture of step 2.
4. Sieve portion of mannitol and add to the mixture of step 3.
5. Blend the mixture of step 4.
6. Sieve remaining portion of mannitol, pre-gelatinised starch, low-substituted hydroxypropyl cellulose and hydroxypropyl cellulose. Add the sieved ingredients to the mixture of step 5.
7. Blend the mixture of step 6.
8. Sieve the blend of step 7.
9. Blend the mixture of step 8.
10. Dissolve hydroxypropyl cellulose in purified water under stirring to form binder solution. Add binder solution to the blend of step 9 and granulate the mass using a high shear granulator (for example Collette).
11. Perform wet screening of mass from step 10 if necessary.
12. Dry the wet granules of step 11 in a fluid bed drier (for example Aeromatic).
13. Screen the dried granules of step 12.
14. Sieve mannitol, low-substituted hydroxypropyl cellulose and talc and add to the sieved granules of step 13.
15. Blend the mixture of step 14.
16. Sieve sodium stearyl fumarate and add to mixture of step 15.
17. Blend the mixture of step 16 to get final blend.
18. Encapsulate the final blend from step 17 using capsule filling machine (for example H&K).

### Description of Manufacturing Process of Form A Formulation A: 25 mg and 75 mg Hard Gelatin Capsules

1. Sieve Form A drug substance, mannitol, pre-gelatinised starch, low substituted hydroxypropyl cellulose, hydroxypropyl cellulose.
2. Blend the sieved materials of step 1.
3. Sieve the mixture of step 2.
4. Blend the mixture of step 3.
5. Dissolve hydroxypropyl cellulose in purified water under stirring to form binder solution. Add binder solution to the blend of step 4 and granulate the mass using a high shear granulator (for example Collette).
6. Perform wet screening of mass from step 6 if necessary
7. Dry the wet granules of step 6 in a fluid bed drier (for example Aeromatic).
8. Screen the dried granules of step 7.
9. Sieve mannitol, low-substituted hydroxypropyl cellulose and talc and add to sieved granules of step 8.
10. Blend the mixture of step 9.
11. Sieve sodium stearyl fumarate and add to step 10.
12. Blend the mixture of step 11 to get final blend.
13. Encapsulate the final blend of step 12.

The processes described above may be reasonably adjusted depending on the available equipment chain and batch scale. Different batch sizes can be prepared by adaptation of equipment size. The weight of individual components for other batch sizes corresponds proportionally to the stated composition within the usual adaptation that may be needed to enable process scale up and transfer as depicted for example in FDA guidance on scale-up and post approval changes.

### Example 7: Further Pharmaceutical Composition Comprising Form A—Formulation ‘B’

Form A was additionally formulated as a hard gelatin capsule (for example Capsugel, size 2 or 3) comprising the ingredients shown in Table 7 (Formulation B). Formulation B manufacture was carried out as described below and in Table 8.

TABLE 7
Unit composition of 10 mg, 15 mg, 25 mg and 50 mg dose strength
formulations of Form A hard gelatin capsules (Formulation B)

| | Formulation B Amount per capsule | | | |
|---|---|---|---|---|
| | (% w/w) | | | |
| | 10 mg | 15 mg | 25 mg | 50 mg |
| Drug load | 8.3% | 8.3% | 20.8% | 20.8% |
| Capsule fill ingredient | | | | |
| Form A | 8.33¹ | 8.33¹ | 20.83¹ | 20.83¹ |
| Mannitol² | 42.97³ | 42.97⁴ | 39.30⁵ | 39.30⁶ |
| Microcrystalline cellulose | 38.83 | 38.83 | 30.00 | 30.00 |
| Low substituted hydroxypropyl | 5.00 | 5.00 | 5.00 | 5.00 |
| cellulose | | | | |
| Hypromellose | 2.87 | 2.87 | 2.87 | 2.87 |
| Sodium stearyl fumarate | 1.50 | 1.50 | 1.50 | 1.50 |
| Talc | 0.50 | 0.50 | 0.50 | 0.50 |
| Purified water⁷ | — | — | — | — |
| Capsule fill weight (mg) | 120.00 | 180.00 | 120.00 | 240.00 |
| Empty capsule shell (theoretical | 48.00⁸ | 61.00⁹ | 48.00⁸ | 61.00⁹ |
| weight in mg) | | | | |
| Total capsule weight (mg) | 168.00 | 241.00 | 168.00 | 301.00 |

¹Formulation B uses a co-milled blend of 50% w/w drug substance and 50% w/w mannitol
²Total mannitol amount in the formulation including mannitol from co-milled blend (pharmaceutical intermediate - PI) and mannitol added in blend for granulation.
³Includes 10.000 mg (8.33% w/w) from co-milled blend and 41.560 mg (34.63% w/w) taken in blend for granulation
⁴Includes 15.000 mg (8.33% w/w) from co-milled blend and 62.340 mg (34.63% w/w) taken in blend for granulation
⁵Includes 25.000 mg (20.83% w/w) from co-milled blend and 22.160 mg (18.47% w/w) taken in blend for granulation
⁶Includes 50.000 mg (20.83% w/w) from co-milled blend and 44.320 mg (18.47% w/w) taken in blend for granulation
⁷Removed during procesing
⁸Formulation B 10 mg (8.33% w/w) and 25 mg (20.83% w/w) dosage strengths are filled in size 3 hard gelatin capsules
⁹Formulation B 15 (8.33% w/w) and 50 mg (20.83% w/w) dosage strength is filled in size 2 hard gelatin capsules

In Formulation B, the Form A drug substance and mannitol are co-milled in order to improve robustness of the milling process. Milling of neat drug substance was found to be challenging due to poor flow and sticking tendency of the material. Examples of suitable mills for the co-milling process include, but are not limited to, Hosokawa Alpine mills, for example: AS, AFG and JS system models; or Fluid Energy Processing & Equipment Company mills, for example: Roto-Jet system models. The co-milled blend is considered as a pharmaceutical intermediate (PI) that is further processed to manufacture the drug product. The co-milled blend utilized in Formulation B contains 50% w/w Form A drug substance and 50% w/w mannitol. Lab scale development trials and small scale pilot manufacturing of co-milled blend containing Form A drug substance up to 70% w/w and mannitol up to 30% w/w (i.e. 70:30—Form A drug substance:mannitol) led to a cumbersome process due to poor material properties of the blend and adherence to the milling chamber. Co-milling of Form A drug substance with 15% w/w mannitol failed. The 50:50% w/w (or 1:1) ratio of Form A drug substance to mannitol was subsequently used based on the positive readout of a manufacturing trial at this ratio.

Formulations A and B are produced by wet granulation technology. Wet granulation was chosen to overcome challenging drug substance physical properties, namely low bulk density, poor flow and wettability. Pregelatinized starch and hydroxypropyl cellulose used as filler and binder respectively in Formulation A were replaced by microcrystalline cellulose and hypromellose. Experiments showed that use of microcrystalline cellulose as filler, rather than pregelatinized starch, led to a faster dissolution profile and improved granule properties. Further experiments showed that use of hypromellose as binder, rather than hydroxypropyl cellulose, provided improved granule properties and granulation process.

TABLE 8
Manufacturing formula for Form A Formulation B: 10 mg, 15 mg, 25 mg
and 50 mg hard gelatin capsules

| Ingredient | Amount per batch (kg) | | | |
|---|---|---|---|---|
| Formulation B dose | 10 mg, | 15 mg, | 25 mg, | 50 mg, |
| strength and batch | 40,000 | 255,650 | 40,000 | 219,000 |
| size | capsules | capsules | capsules | capsules |
| Capsule fill | | | | |
| Form A PI¹ | 0.800 | 7.670 | 2.000 | 21.900 |
| Microcrystalline | 1.864 | 17.870 | 1.440 | 15.768 |
| cellulose | | | | |
| Mannitol | 1.662 | 15.937 | 0.886 | 9.706 |
| Low substituted | 0.240 | 2.301 | 0.240 | 2.628 |
| hydroxypropyl cellulose | | | | |
| Hypromellose | 0.138 | 1.319 | 0.138 | 1.507 |
| Sodium stearyl fumarate | 0.072 | 0.690 | 0.072 | 0.788 |
| Talc | 0.024 | 0.230 | 0.024 | 0.263 |
| Purified water² | q.s | q.s | q.s | q.s |
| Weight capsule fill mix | 4.800 | 46.017 | 4.800 | 52.560 |
| Empty capsule shell | 1.920 | 15.595 | 1.920 | 13.359 |
| Capsule shell³ | | | | |
| (theoretical weight) | | | | |
| Total batch weight | 6.720 | 61.612 | 6.720 | 65.919 |

¹If PI drug content is ≤99.5% or ≥100.5%, the weight will be adjusted and compensated with mannitol
²Removed during processing
³10 and 25 mg dose strength blends were filled into Size 3 hard gelatin capsules whereas 15 and 50 mg does strength blends were filled into Size 2 hard gelatin capsules
q.s = quantum satis (to be added as needed)

Table 8 provides the ingredients for particular batch sizes. Other batch sizes may be utilised depending on clinical requirements and/or available equipment and/or available starting materials. The weight of individual components for other batch sizes corresponds proportionally to the stated composition.

### Description of Manufacturing Process

The process described below may be reasonably adjusted, while maintaining the same basic production steps, to compensate for different batch sizes and/or equipment characteristics, and/or on the basis of experience of the previous production batch.

### PI Manufacture

1. Blend Form A drug substance and mannitol.
2. Sieve the blend of step 1.
3. Co-mill the sieved material of step 2.
4. Blend the co-milled material of step 3 to obtain Form A PI

### Form A Formulation B: 15 mg and 50 mg Hard Gelatin Capsules

1. Sieve Form A PI, mannitol, microcrystalline cellulose, and low substituted hydroxypropyl cellulose.
2. Blend the sieved materials of step 1.
3. Sieve the mixture of step 2.
4. Blend the mixture of step 3.
5. Dissolve hypromellose in purified water under stirring to form binder solution. Add binder solution to the blend of step 4 and granulate the mass using a high shear granulator (for example Collette Model GRAL). Add additional purified water if necessary. Target amount of total water: approximately 25%.
6. Perform wet screening based on visual observation/assessment of wet granules of step 5 (optional).
7. Dry the wet granules of step 6 in a fluid bed dryer (for example Aeromatic).
8. Screen the dried granules of step 7.
9. Sieve low-substituted hydroxypropyl cellulose and talc and add to sieved granules of step 8.
10. Blend the mixture of step 9.
11. Sieve sodium stearyl fumarate and add to step 10.
12. Blend the mixture of step 11 to get final blend.
13. Encapsulate the final blend of step 12 into hard gelatin capsules.

### Example 8: Crystallisation Procedure for Form B

3.5 g of Form A was suspended in 5 ml of THF in a 20 ml glass vial. The suspension was stirred with 300 rpm at room temperature for one week. The suspension was filtrated by centrifuge filtration tube and the solid was dried at room temperature overnight to yield about 1.39 g of Form B.

### Example 9: XRPD Analysis of Form B

Crystalline Form B was analysed by XRPD and the ten most characteristic peaks are shown in Table 9 (see also FIG. 4).

TABLE 9

| 2-theta in degrees | relative intensity in % |
|---|---|
| 10.65 | 68.3 |
| 13.48 | 100.0 |
| 16.56 | 29.9 |
| 16.77 | 47.1 |
| 17.39 | 18.6 |
| 19.71 | 17.0 |
| 20.45 | 22.8 |
| 21.34 | 55.9 |
| 23.08 | 18.0 |
| 27.16 | 20.7 |

### Example 10: DSC Analysis of Form B

Form B was analysed by differential scanning calorimetry (DSC) using a Discovery Diffraction Scanning Calorimeter from TA instruments and shows an onset of conversion at about 119° C. due to transformation into Form A, followed by an onset of melting at about 170° C., consistent with Form A, see FIG. 5. Heating rate was performed at 10° C. per minute.

### Example 11: Crystallisation Procedure for Hemihydrate Form H_{A}

2.5 g of Form A was dissolved in a mixture of 15 ml IPAc/0.375 ml water at 65° C. with 300 rpm stirring (stir bar). The clear solution was cooled to 45° C. over 20 min. 7 ml of n-heptane was added dropwise to the solution by injection pump over 10 min at 500 rpm (stir bar). The solution was cooled to 15° C. over 2 h. 20 ml of n-heptane was added over 50 min and then 37 ml of n-heptane was added over 93 min at 200 rpm (paddle). The suspension was stirred overnight (at least 10 h) at 15° C., filtered and washed with n-heptane. The solid was dried at room temperature. 1.78 g of hemihydrate form H_{A} was obtained.

### Example 12: XRPD Analysis of Hemihydrate Form H_{A}

Crystalline Hemihydrate Form H_{A} was analysed by XRPD and the ten most characteristic peaks are shown in Table 10 (see also FIG. 6).

TABLE 10

| 2-theta in degrees | relative intensity in % |
|---|---|
| 13.96 | 65.0 |
| 14.28 | 100.0 |
| 16.07 | 39.0 |
| 17.66 | 47.6 |
| 18.33 | 83.9 |
| 19.57 | 38.2 |
| 21.40 | 30.3 |
| 21.56 | 34.0 |
| 24.06 | 28.8 |
| 25.83 | 25.8 |

### Example 13: DSC Analysis of Hemihydrate Form H_{A}

Form H_{A} was analysed by differential scanning calorimetry (DSC) using a Discovery Diffraction Scanning Calorimeter from TA instruments and found to have an onset of dehydration temperature at about 98° C., followed by recrystallization and an onset of melting at 170° C., consistent with Form A. Heating rate was performed at 10° C. per minute (pierced pan), see FIG. 7.

### Example 14: Preparation of Amorphous Form

2.0 g of Form A was dissolved in 100 ml of 1,4-dioxane and frozen by acetone dry ice bath. The sample was freeze-dried for 1 day and then characterized by XRPD. No diffraction peak was observed. The solid was dried in a vacuum oven at 70° C. for 2 hours and then was stored at −20° C. See FIG. 8.

### Example 15: DSC Analysis of Amorphous Form

Amorphous form was analysed by means of mDSC using a Discovery Diffraction Scanning Calorimeter from TA instruments at 2K/min from −20 to 200° C. with modulate temperature amplitude 1K and period of 60 s. A glass transition can be detected at about 59° C., followed by recrystallisation. The melting point of the resulting form is consistent with that of Form A, see FIG. 9.

### Example 16: Chemical Stability of Crystalline Form A when Exposed to High Temperature/Humidity for One Week

The stability of crystalline Form A was tested by exposing the crystalline material to high temperature and/or humidity for at least three weeks. After storage at high temperature and/or humidity, bulk crystalline material was sampled and dissolved in acetonitrile:water (80:20) and the purity analysed in a Waters Aquity UPLC using the following conditions:

TABLE 11

| Separation column | Waters Acquity UPLC BEH Phenyl | | |
|---|---|---|---|
| Mobile phase | A: 0.05% TFA in 95% water/5% acetonitrile; | | |
| | B: 0.05% TFA in 95% acetonitrile/5% water | | |
| Flow rate | 0.6 mL/min | | |
| Column Temperature° | 35° C. | | |
| Detection | 286 nm | | |
| | Time (min) | % A | % B |
| Gradient | 0.0 | 95 | 5 |
| | 2.5 | 60 | 40 |
| | 3.5 | 54 | 46 |
| | 5.0 | 5 | 95 |
| | 5.01 | 95 | 5 |
| | 6.0 | 95 | 5 |

The results of this test are shown in Table 12 below.

TABLE 12

| Test Conditions | | |
|---|---|---|
| Temp/RH; Exposure Time | Purity/% | Solid State Form |
| RT; 0 | 97.3 | Crystalline |
| 80° C.; 3 weeks | 97.3 | Crystalline |
| 50° C.; 4 weeks | 97.3 | Crystalline |
| 50° C./75% RH; 3 weeks | 96.8 | Crystalline |

The stability data of Form A, outlined in Table 12, was compared to Form B, hydrate Form H_{A} and the amorphous form, tested under the same conditions, and in each case conversion into Form A occurred, indicated by XRPD analysis. Form A was found to be the most thermodynamically stable polymorph.

### Example 17: In Human Study of Pharmacokinetics of Free Base Compound 1 when Given Alone and in Combination with the Strong CYP3A4 Inhibitor Itraconazole or the Strong CYP3A4 Inducer Rifampicin

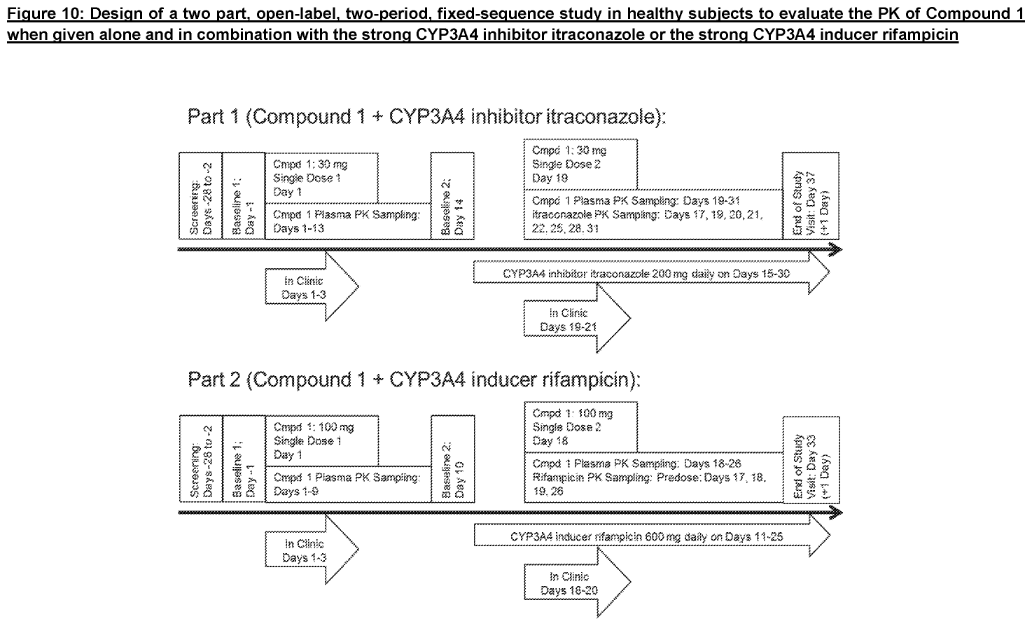

In a drug-drug interaction (DDI) study in healthy volunteers, the effect of a strong CYP3A4 inhibitor (itraconazole) and a strong CYP3A4 inducer (rifampicin) on the PK of Compound 1 was evaluated. The DDI study design is outlined in FIG. 10. Itraconazole, at a dose of 200 mg q.d., increased mean AUC of Compound 1 2-3-fold and mean Cmax of Compound 1 by 25%, when given together with Compound 1 as compared to when Compound 1 was given alone (Table 13). Rifampicin, at a dose of 600 mg q.d., decreased mean AUC of Compound 1 5-6-fold and mean Cmax of Compound 1 2.5-fold, when given together with Compound 1 as compared to when Compound 1 was given alone (Table 14). In conclusion, the effect of a strong CYP3A4 inducer and a strong CYP3A4 inhibitor on Compound 1 exposure in a Phase 1 study has shown that CYP3A4 is of major importance for the elimination of Compound 1 and that the effects of co-treatment with a strong CYP3A4 inhibitor or inducer need to be taken into account when administering Compound 1.

TABLE 13
Pharmacokinetic results - Statistical analysis of the effect of itraconazole on the
plasma PK parameters of Compound 1: Compound 1 30 mg SD + itraconazole 200 mg
QD vs Compound 1 30 mg SD

| | | | Adjusted | Geometric mean | |
|---|---|---|---|---|---|
| Parameter | | | geometric | ratio | 90% CI for |
| [Unit] | Treatment | n* | mean | (Test/Reference) | ratio |
| AUCinf | Cmpd 1 30 mg SD | 17 | 3560 | 3.05 | [2.91, 3.20] |
| (ng * hr/mL) | Cmpd 1 30 mg SD + | 17 | 10900 | | |
| | Itraconazole 200 mg | | | | |
| | QD | | | | |
| AUClast | Cmpd 1 30 mg SD | 17 | 3150 | 2.20 | [2.11, 2.30] |
| (ng * hr/mL) | Cmpd 1 30 mg SD + | 17 | 6930 | | |
| | Itraconazole 200 mg | | | | |
| | QD | | | | |
| Cmax | Cmpd 1 30 mg SD | 17 | 74.1 | 1.23 | [1.18, 1.29] |
| (ng/mL) | Cmpd 1 30 mg SD + | 17 | 91.3 | | |
| | Itraconazole 200 mg | | | | |
| | QD | | | | |

n* = number of subjects with non-missing values.
An ANOVA model with fixed effects for treatment and subject was fitted to each log-transformed PK parameter. Results were back transformed to obtain ‘Adjusted geo-mean’, ‘Geo-mean ratio’ and ‘90% CI’.

TABLE 14
Pharmacokinetic results - statistical analysis of the effect of rifampicin on the
plasma PK parameters of Compound 1: Compound 1 100 mg SD + rifampicin 600 mg
QD vs Compound 1 100 mg SD

| | | | Adjusted | Geometric mean | |
|---|---|---|---|---|---|
| Parameter | | | geometric | ratio | |
| [Unit] | Treatment | n* | mean | (Test/Reference) | 90% CI for ratio |
| AUCinf | Cmpd 1 100 mg SD | 13 | 10200 | 0.172 | [0.152, 0.194] |
| (ng * hr/mL) | Cmpd 1 100 mg SD + | 13 | 1750 | | |
| | Rifampicin 600 mg QD | | | | |
| AUClast | Cmpd 1 100 mg SD | 13 | 8560 | 0.196 | [0.176, 0.219] |
| (ng * hr/mL) | Cmpd 1 100 mg SD + | 13 | 1680 | | |
| | Rifampicin 600 mg QD | | | | |
| Cmax | Cmpd 1 100 mg SD | 13 | 222 | 0.414 | [0.365, 0.470] |
| (ng/mL) | Cmpd 1 100 mg SD + | 13 | 92.2 | | |
| | Rifampicin 600 mg QD | | | | |

n* = number of subjects with non-missing values.
An ANOVA model with fixed effects for treatment and subject was fitted to each log-transformed PK parameter. Results were back transformed to obtain ‘Adjusted geo-mean’, ‘Geo-mean ratio’ and ‘90% CI’.

### REFERENCES

Charidimou A et al. (2011) Sporadic cerebral amyloid angiopathy revisited: recent insights into pathophysiology and clinical spectrum. J. Neurol. Neurosurg. Psychiatry; 83(2):124-137.
Chen Y W et al. (2006) Progression of white matter lesions and hemorrhages in cerebral amyloid angiopathy. Neurology; 67(1):83-87.
Cheng A L et al. (2013) Susceptibility-Weighted Imaging is More Reliable Than T2*-Weighted Gradient-Recalled Echo MRI for Detecting Microbleeds. Stroke; 44(10):2782-2786.
Gennaro, A R (1990) Remington's Pharmaceutical Sciences. 18th Ed. Mack Printing Company, pp. 1289-1329.
Greenberg S M et al. (2014) Outcome Markers for Clinical Trials in Cerebral Amyloid Angiopathy. Lancet Neurol.; 13(4):419-428.
Gurol M E et al. (2016) Florbetapir-PET to diagnose cerebral amyloid angiopathy: A prospective study. Neurology; 87(19):2043-2049.
Kramp V P, Herrling P (2011) List of drugs in development for neurodegenerative diseases: Update June 2010. Neurodegenerative Dis.; 8:44-94.
Midoux N et al. (1999) Micronization of pharmaceutical substances in a spiral jet mill. Powder Technol.; 104:113-120.
Mullin J W, Nyvlt J (1971) Programmed cooling of batch crystallizers. Chem. Eng. Science; 26(3): 369-377.
Quintino-Santos S R et al. (2012) Homozygosity for the APOE E4 allele is solely associated with lower cognitive performance in Brazilian community dwelling older adults—The Bambui Study. Rev. Bras. Psiquiatr.; 34(4):440-445.
Shinohara M et al. (2016) Impact of sex and APOE4 on cerebral amyloid angiopathy in Alzheimer's disease. Acta Neuropathol.; 132(2):225-234.
Sperling R A et al. (2011) Toward defining the preclinical stages of Alzheimer's disease: Recommendations from the National Institute on Aging and the Alzheimer's Association workgroup. Alzheimers Dement.; 7(3):1-13.
Thal D R et al. (2002) Two types of sporadic cerebral amyloid angiopathy. J. Neuropathol. Exp. Neurol.; 61: 282-293.
Verghese P B et al. (2011) Roles of Apolipoprotein E in Alzheimer's disease and Other Neurological Disorders. Lancet Neurol.; 10(3): 241-252.

All references, for example, a scientific publication or patent application publication, cited herein are incorporated herein by reference in their entirety and for all purposes to the same extent as if each reference was specifically and individually indicated to be incorporated by reference in its entirety for all purposes. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A crystalline Form A of the compound

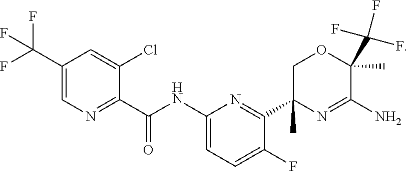 [SMILES: C[C@@]1(c2nc(NC(=O)c3ncc(C(F)(F)F)cc3Cl)ccc2F)CO[C@@](C)(C(F)(F)F)C(N)=N1]

2. The crystalline Form A of the compound according to claim 1 in substantially pure form.

3. The crystalline Form A of the compound according to claim 1 which has an X-ray powder diffraction pattern with at least one, two or three peaks having angle of refraction 2 theta (θ) values selected from 10.7, 14.8 and 19.50 when measured using CuKα radiation, wherein said values are plus or minus 0.2° 2θ.

4. The crystalline Form A of the compound according to claim 1 which has an X-ray powder diffraction pattern with at least one, two or three peaks having angle of refraction 2 theta (θ) values selected from 14.8, 18.7 and 19.5° when measured using CuKα radiation, wherein said values are plus or minus 0.2° 2θ.

5. The crystalline Form A of the compound according to claim 1 which has an X-ray powder diffraction pattern with at least one, two or three peaks having angle of refraction 2 theta (θ) values selected from 10.7, 14.8, 18.7, 19.5 and 21.40 when measured using CuKα radiation, wherein said values are plus or minus 0.2° 2θ.

6. The crystalline Form A of the compound according to claim 1 which has an X-ray powder diffraction pattern with at least one, two, three, four or five peaks having angle of refraction 2 theta (θ) values selected from 10.7, 14.8, 18.7, 19.5, 21.4, 21.7, 25.5, 29.9, 35.0, 37.8° when measured using CuKα radiation, wherein said values are plus or minus 0.2° 2θ.

7. The crystalline Form A of the compound according to claim 1 which has an X-ray powder diffraction pattern substantially the same as the X-ray powder diffraction pattern shown in FIG. 1 when measured using CuKα radiation.

8. The crystalline Form A of the compound according to claim 1 in micronized form, which has an X-ray powder diffraction with at least one, two or three peaks having angle of refraction 2 theta (θ) values selected from 12.1, 19.4 and 24.0° when measured using CuKα radiation, wherein said values are plus or minus 0.2° 2θ.

9. The crystalline Form A of the compound according to claim 1 in micronized form, which has an X-ray powder diffraction pattern with at least one, two or three peaks having angle of refraction 2 theta (θ) values selected from 12.1, 15.9, 18.5, 19.4, 24.00 when measured using CuKα radiation, wherein said values are plus or minus 0.2° 2θ.

10. The crystalline Form A of the compound according to claim 1 in micronized form, which has an X-ray powder diffraction pattern with at least one, two, three, four or five peaks having angle of refraction 2 theta (θ) values selected from 10.6, 12.1, 14.7, 15.9, 18.5, 19.4, 21.2, 24.0, 24.7, 29.7° when measured using CuKα radiation, wherein said values are plus or minus 0.2° 2θ.

11. The crystalline Form A of the compound according to claim 1 in micronized form, which has an X-ray powder diffraction pattern substantially the same as the X-ray powder diffraction pattern shown in FIG. 3 when measured using CuKα radiation.

12. The crystalline Form A of the compound according to claim 1 having a differential scanning calorimetry (DSC) thermogram substantially the same as that shown in FIG. 2.

13. A pharmaceutical composition comprising the crystalline Form A of the compound according to claim 1 and at least one pharmaceutically acceptable carrier or diluent.

14. (canceled)

15. A method for treating or preventing Alzheimer's disease comprising the crystalline form of the compound according to claim 1.
